SEQUENCE LISTING (12 sequences)

SEQ ID NO: 1 - protein, 10 aa, Artificial sequence
  Synthetic construct
KHKHKHKHKK

SEQ ID NO: 2 - protein, 153 aa, Homo sapiens
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAK

SEQ ID NO: 3 - protein, 66 aa, Artificial Sequence
  Synthetic construct
KHKHKHKHKKCKHKHKHKHKKCKHKHKHKHKKCKHKHKHKHKKCKHKHKHKHKKCKHKHKHKHKKC

SEQ ID NO: 4 - protein, 185 aa, Artificial Sequence
  Synthetic construct
KHKHKHKHKKKHKHKHKHKKKHKHKHKHKKMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 215 aa, Artificial Sequence
  Synthetic construct
KHKHKHKHKKKHKHKHKHKKKHKHKHKHKKKHKHKHKHKKKHKHKHKHKKKHKHKHKHKKMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 6 - protein, 188 aa, Artificial Sequence
  Synthetic construct
KHKHKHKHKKCKHKHKHKHKKCKHKHKHKHKKCMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 7 - DNA, 96 bp, Artificial Sequence
  Synthetic construct
agttaggatccctcttcaaagcacaaacataagcacaagcacaagaagaaacataaacacaagcataaacacaaaaagtgaagaggaattctaact

SEQ ID NO: 8 - protein, 11 aa, Artificial sequence
  Synthetic construct
KHKHKHKHKKC

SEQ ID NO: 9 - protein, 7 aa, Artificial sequence
  Synthetic construct
KHKHCKK

SEQ ID NO: 10 - DNA, 28 bp, Artificial sequence
  Synthetic construct
gttccacatatggcagccgggagcatca

SEQ ID NO: 11 - DNA, 30 bp, Artificial sequence
  Synthetic construct
cgggaaaagcttgctcttagcagacattgg

SEQ ID NO: 12 - protein, 185 aa, Artificial Sequence; identical to SEQ ID NO: 4
  Synthetic construct

(19) United States
(12) Patent Application Publication
(10) Pub. No.: US 2007/0098702 A1
(43) Pub. Date: May 3, 2007

(54) Recombinant protein polymer vectors for systemic gene delivery

(75) Inventors: Zaki Megeed, Cambridge, MA (US); Arash Hatefi, Timonium, MD (US); Hamidreza Ghandehari, Ellicott City, MD (US)

Correspondence Address: EVANS & MOLINELLI, PLLC, U.S. POST OFFICE BOX 7024, FAIRFAX STATION, VA, 22039, US

(73) Assignee: University of Maryland, Baltimore

(21) Appl. No.: 11/357,499

(22) Filed: Feb. 17, 2006

Related U.S. Application Data
(60) Provisional application No. 60/654,015, filed on Feb. 17, 2005.

(51) Int. Cl. A61K 48/00; C12N 15/82; C12N 15/74

(52) U.S. Cl. 424/93.21; 435/455; 435/468; 435/471

(57) ABSTRACT

The present invention relates to genetically engineered non-viral vectors for delivering a nucleic acid such as a therapeutic gene to a target cell. The vectors are suitable for systemic administration to an animal. In the simplest embodiment the non-viral vector is a nucleic acid-binding protein-based polymer (NABP) having at least one tandem repeat of a genetically engineered cationic amino acid-containing monomer (CAACM) containing lysine, arginine or a combination thereof, which confers on the NABP the ability to bind a nucleic acid that is intended for delivery to a target cell. Because the NABP is genetically engineered and transcribed from a single gene, its structure and function can be precisely controlled. The vectors optionally have additional functionalities including endosome disrupting moieties, targeting ligands and subcellular localization sequences.

### CROSS-REFERENCE TO RELATED APPLICATIONS

This application claims benefit of Provisional Application. 60/60/654,015, filed on Feb. 17, 2005, the entire contents of which are hereby incorporated by reference as if fully set forth herein, under 35 U.S.C. §119(e).

### EXTENT OF GOVERNMENTAL INTEREST

This invention was made with Government support under Grant No. DAMD17-03-1-0534 awarded by the Department of the Defense. The Government has certain rights in the invention.

### BACKGROUND OF THE INVENTION

1. Field of the Invention

The present invention relates to genetically engineered amino acid based non-viral vectors for gene therapy.

2. Description of the Related Art

A major obstacle for successful gene therapy for cancer and other diseases has been the unavailability of safe and clinically effective gene delivery systems [1]. For genetic material to successfully reach the target site upon systemic administration it must be protected from degradation by nucleases, targeted to specific cell types of interest, internalized by the targeted cell with high transfection efficiency, escape from the endosomes, dissociate from the carrier, enter the nucleus, and finally be expressed. Through evolutionary processes viruses have developed means to overcome these barriers. As a result viral vectors in the past have offered superior transfection efficiency in comparison to non-viral vectors. However, their clinical use has been plagued by concerns about their safety.

Until now, non-viral gene delivery research has focused substantially on the chemical synthesis and characterization of new vectors such as poly amino acids, lipids, and peptides [2-4]. Synthetic vectors such as polymers have the potential to reduce the safety problems associated with viral vectors; however their low transfection efficiency limits their clinical utility. Polymeric amino acid carriers that have been made for gene delivery in the past were all synthesized using traditional chemical synthetic methods, which results in the production of polymers with random sequences and variation in molecular weight making it difficult to attach functional motifs at precise locations such as targeting ligands, EDM and NLS [5-7].

Some polymers for use as vectors have been made from sequential poly peptides and random copolymers of poly amino acids. Sequential poly peptides are made from chemical polymerization of blocks of amino acids that are synthesized by solution or solid phase synthesis, and the number of amino acids that can be incorporated in each monomer block is limited. Further there is an uneven distribution of molecular weights upon polymerization of the monomer blocks, and side reactions such as racemization are also common [8]. Poly amino acids made from random copolymerization of two or more amino acids offer less control over sequence and length, and little control over the final copolymer composition.

Transfection and gene expression in various cell lines using chemically synthesized poly lysine as a non-viral vector has been studied. The cell lines used include HepG2 hepatoblastoma, P388D1 macrophage cell line to approximate transfection of antigen-presenting cells for DNA-based vaccines, and the CRL 1476 muscle cell line used to mimic muscle transfection after intramuscular injection [9]. These studies showed that poly cations like polylysine can condense DNA, deliver it to target cells and achieve significant gene expression. The DNA in these experiments condensed into toroidal nanostructures suitable for gene delivery with a size less than 150 nm. Some non-viral chemically synthesized polymeric and peptidic delivery systems used in the past include polylysine and copolymers thereof [10-19].

Inherent in these studies are the problems of randomization that occurs with chemical synthesis. However, directed synthesis of polymers/copolymers with repeats of cationic amino acids used to make these polymers does not permit control over long-range sequence; only short peptide chains can be synthesized. Further, stereochemistry is difficult to control with directed synthesis, and the final polymers are still polydisperse.

Without full control over the size and composition of the polymers/copolymers, vector efficiency and consistency are seriously compromised [6, 7, 9, 20]. Therefore there is a great need for a new method to make non-viral vectors using genetic engineering that have precise, consistent, and predictable structures to facilitate predictable binding (or condensation) of the therapeutic gene and delivery to a target cell.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1. A) Overview of the cloning strategy used to fuse (KH)₆ gene along with FGF2 gene in pET21b expression vector. B) Primary sequence of (KH)₆-FGF2 based on DNA sequencing results. The lysine-histidine repeats are shown in bold whereas the FGF2 sequence is underlined. The position of his-tag at C-terminal is also demonstrated. Theoretical pI/Mw: 10.13/27,313.

FIG. 2. SDS-PAGE and western blot analysis of purified (KH)₆-FGF2. A) SDS-PAGE of purified (KH)₆-FGF2 with >95% purity; B) Western blot analysis of purified (KH)₆-FGF2 using Anti 6× His antibody recognizing 6 sequential histidine residues at the C-terminal of the expressed vector. M stands for the protein Marker.

FIG. 3. A) Agarose gel electrophoresis of the DNA/vector complexes. All the complexes were prepared in 5 mM PBS, and subsequently 10% v/v serum was added to the complexes. 1) DNA alone, 2) DNA+serum, 3) DNA to vector 1:40 mole/mole, 4) DNA to vector 1:60 mole/mole, 5) DNA to vector 1:80 mole/mole, 6) DNA to vector 1:100 mole/mole. B) Agarose gel electrophoresis of DNA/vector complexes in 5 mM phosphate buffer at various mole/mole ratios (no serum). 1) pDNA (control), 2) 1:50 mole/mole, 3) 1:100 mole/mole, 4) 1:150 mole/mole, 5) 1:200 mole/mole.

FIG. 4. A) WST-1 cell proliferation assay for NIH 3T3 cells treated with (KH)₅ (open bars), (KH)₆-FGF2 (light grey bars) and FGF2 (closed bars). Cells were treated with various concentrations ranging from 0 (control) to 50 ng/ml and the absorbance of soluble formazan was measured at 440 nm (Mean±S.D., n=4). B) WST-1 cell toxicity assay for NIH 3T3 cells treated with various concentrations of (KH)₅ (open bars) and (KH)₆-FGF2 (light grey bars) ranging from 0 to 50 μg/ml. No significant toxicity was observed in either case.

FIG. 5. Percentage of cells transfected with (KH)₆-FGF2/pEGFP complexes (Mean±S.D., n=9). Closed bars: Cells transfected in serum free media. Open bars: Cells transfected in growth media containing serum.

FIG. 6. Competitive inhibition assay showing cell transfection via FGF2 receptor-mediated endocytosis. A) Confocal microscopy image of NIH 3T3 cells transfected with (KH)₆-FGF2/pEGFP in serum free media, B) Confocal microscopy image of NIH 3T3 cells transfected with (KH)₆-FGF2/pEGFP in serum free media with addition of 1000 ng/ml FGF2, C) Percentage of cells transfected in SFM vs SFM+FGF2.

FIG. 7. The full oligonucleotide sequence of the sense strand designed for multimerization of a KH monomer with pertinent restriction sites. Starting from 5′: Bam HI recognition site (bold), Eam 1104 I recognition site (underlined), nucleotides encoding KH monomer, Eam 1104 I recognition site (underlined), Eco RI recognition site (bold). The (KH) monomer is (KHKHKHKHKK) SEQ ID NO. 1.

FIG. 8. Polyacrylamide gel electrophoresis on amplified monomer cut with Eam 1104 I. Lanes 1 and 2 contain monomer cut with 1× and 10× enzyme concentrations. Bottom band (faint, arrow) is the digested monomer. Central two bands are amplified monomer digested on one side. Top band is undigested amplified monomer.

FIG. 9. Production of (KH) DNA concatemers. A) The KH monomer (The (KH) monomer (KHKHKHKHKK) is first cut from pZero-2 with Eam 1104 I.). B) After purification by gel electrophoresis, monomer is obtained. C) Concatemerization with T4 DNA ligase yields concatemers with different lengths. In A, B and C, left lanes are DNA markers.

FIG. 10. Example of PCR colony screening result after screening E. coli TOP10 transformed with pAAG containing KH concatemers. Various concatemeric sequences with different molecular weights are obtained. Sequences in lanes 2 and 8 were chosen for ligation with FGF2 gene and further expression. M=DNA molecular weight markers.

FIG. 11. Amino acid sequence of Low Molecular Weight-FGF2, SEQ ID NO. 2.

FIG. 12. PCR primer sequences for FGF2 gene amplification. SEQ ID NO. 10 and SEQ ID NO. 11.

FIG. 13. 1% Agarose gel electrophoresis of amplified FGF2 gene. From left to right, DNA Ladder 1 kbp, DNA Ladder 100 bp and amplified gene corresponding to 480 bp FGF2 gene.

FIG. 14: Western blot analysis of expressed FGF2 gene using Anti-FGF2 as the primary antibody. From right to left: A) protein marker, B) BL21 (DE3) cells uninduced, C) BL21 (DE3) cells induced T=1, D) BL21 (DE3) cells induced T=2, E) BL21 (DE3) cells induced T=3, F) BL21 (DE3) cells induced T=4.

FIG. 15. Western Blot analysis of expressed (KH)₆-FGF2 gene using anti-FGF2 as the primary antibody. From right to left: A) protein marker, B) BL21 (DE3) cells uninduced, C) BL21 (DE3) cells induced T=1, D) BL21 (DE3) cells induced T=2, E) BL21 (DE3) cells induced T=3, F) BL21 (DE3) cells induced T=4.

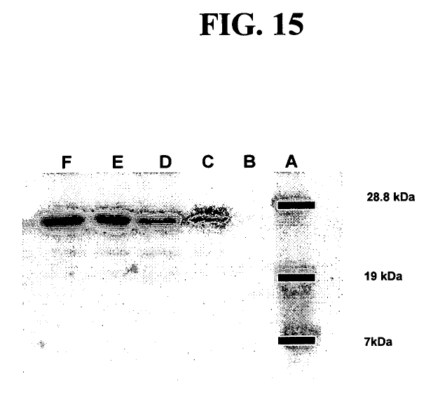

### DEFINITIONS

Non-viral Vector: “Non-viral” means a group of a variety of unrelated structures such as protein based polymers (also referred to herein as amino acid polymers), linear and branched polycations, block copolymers, intact and fractured dendrimers, nanospheres, polysaccharides, cationic liposome formulations, modular fusion proteins, and peptides.

Genetically Engineered Polymer: means a polymer made of multiple repeating amino acid residues that is transcribed from a single gene, in a sequence found anywhere in nature or any artificial sequence that contains a polymer region.

Nucleic acid-binding protein-based polymer (NABP or also referred to herein as an amino acid-based polymer): means a genetically engineered polymer transcribed from a single gene that has at least one tandem repeat of a cationic amino acid-containing monomer as herein defined. The NABP has amino acids that are positively charged at pH 7.4 that bind to negatively charged nucleic acids at this pH. The non-viral vectors of the present invention all include a nucleic acid-binding protein-based polymer region.

Nucleic Acids: is used broadly to include a therapeutic gene or other gene, DNA, RNA including antisense-RNA and small-interfering RNA, and oligonucleotides.

Cationic amino acid-containing monomer (CAACM): means a nucleic acid-binding monomer that contains lysine or arginine, or a combination thereof. Lysine and arginine are cationic amino acids that are positively charged at pH 7.4 thus enabling them to bind negatively charged nucleic acids for delivery to a target cell. The cationic amino acid histidine (H) is not positively charged at pH 7.4 because the pKa of histidine is about 6. However, histidine is often included in CAACM because it disrupts endosomes facilitating the release of the nucleic acid/vector complex from the endosome. Lysine and/or arginine are often repeated more than once in a CAACM, typically separated by an amino acid that is not positively charged at pH 7.4, such as histidine or the non-cationic amino acids. Examples of CAACM include (KHKHKHKHKK), (RHRHKHC), and (KHKHCKK) SEQ ID NO. 9, (KGKGRC). Lysine and/or arginine can be used in any combination with or without other amino acids to make the CAACM.

“Tandem repeat”: means at least one identical CAACM after another in tandem within the nucleic acid-binding protein-based polymer (NABP) also identified as (monomer)n, where n is 2 to 100. One example of a tandem repeat of a CAACM in an NABP is (KHKHKHKHKK)₂—(KHKHCKK). In this example, the first segment of the polymer has a single tandem repeat of the (KHKHKHKHKK) monomer while the CAACM (KHKHCKK) in the second segment is not repeated. There can be more than one tandem repeat of the monomer in an NABP, for example (KHKHKHKHKK)₆ or (KKKKK)₃—(KCKKH)₂.

Nucleic Acid Binding Moiety (NABM): means that portion of a non-viral vector of the present invention that bind to the nucleic acid intended for delivery to a target cell, namely the nucleic acid-binding protein-based polymer (NABP).

Concatemer: means a DNA segment composed of repeated nucleotide sequences linked end to end in a single gene.

Targeting Ligand: means any molecular signal directing localization to specific cells, tissues, or organs. Proteins that bind to cell surface receptors come within the definition of targeting ligand as do antibodies directed to antigens expressed selectively on a target cell.

Nuclear Localization Signal (NLS): means any compound capable of facilitating the active nuclear import and/or export of proteins from the nucleus. Typically NLS are amino-acid sequences, often having basic amino acids, but the term for the purpose of this invention is not so limited. Any protein or peptide facilitating the active nuclear import and/or export of proteins is an NLS for the purpose of this invention.

Endosome Disrupting Moiety: means any protein or peptide capable of disrupting or lysing the endosome membrane resulting in release of the endosomal content; it is usually a sequence of amino acids.

Target Cells: means any eukaryotic or prokaryotic cell intended as the recipient cell for delivery of a nucleic acid, including any animal cell whether normal or diseased such as a cancer cell, bacterial and plant cells.

Trash amino acid: means any amino acid other than the amino acid sequence of the protein of interest. The trash amino acids are usually introduced during the cloning of the genes into cloning vectors to facilitate gene cloning, protein purification, and detection. For example, fusion of 6 histidine at the C-terminal or N-terminal of a protein facilitates protein purification by Ni-column chromatography. In many cases to facilitate the fusion of different genes which encode two or more different proteins, new restriction sites need to be strategically placed in between the genes. These nucleotide sequences which are recognized by different restriction enzymes will in turn translate into amino acids which are not part of the protein of interest and considered trash amino acids.

### SUMMARY OF THE INVENTION

Some embodiments of the invention include a genetically engineered non-viral vector for delivering a nucleic acid molecule to a target cell, made of a nucleic acid-binding protein-based polymer that has at least one tandem repeat of a cationic amino acid-containing monomer (CAACM) which monomer is capable of binding to the nucleic acid molecule. In some embodiments the CAACM has one or more amino acids selected from the group consisting of lysine and arginine. In another embodiment the cationic amino acid-containing monomer further comprises histidine, which also serves as an endosome disrupting moiety. Some other embodiments are directed to vectors where the CAACM contains one or more cysteine residues. In some embodiments the cationic amino acid-containing monomer is a homopolymer of lysine or arginine.

In some embodiments the vectors are multifunctional and in addition to the nucleic acid binding protein-based polymer (NABP) the vectors also have a protein or peptide targeting ligand that is recognized by a target cell, such as a ligand that binds to a cell receptor, such as on a cancer cell, or an antibody that recognizes an antigen on the surface of the target cell. In some embodiments the vectors have fibroblast growth factor 2 (FGF2) or a fragment thereof as a targeting ligand. The target cell can be a plant, bacterial or animal cell.

In other embodiments the vectors have in addition to the NABP, a nuclear localization sequence or an endosome disrupting moiety. In one embodiment the vectors have NABP, targeting land, a nuclear localization sequence and an endosome disrupting moiety. The vectors of the present invention are transcribed from a single gene.

In other embodiments the vector is bound to a nucleic acid molecule that can be DNA or RNA, forming a vector/nucleic acid complex. One embodiment is directed to a pharmacological composition for gene therapy, having a genetically engineered vector as described above bound to a therapeutic gene. In one further embodiment the vector in the complex has a targeting ligand, such as FGF2, that is recognized by a target cell including a cancer cell. In another embodiment the therapeutic gene is delivered to a cancer cell.

Certain embodiments include a method for delivering a nucleic acid molecule to a target cell, by a) obtaining a genetically engineered non-viral vector comprising a nucleic acid-binding protein-based polymer that contains at least one tandem repeat of a cationic amino acid-containing monomer capable of binding to the nucleic acid molecule; b) contacting the vector of step a with the nucleic acid molecule under conditions that permit the vector to bind to the nucleic acid molecule to form a complex; and c) contacting the vector/nucleic acid molecule complex of step b with the target cell under conditions that permit the vector/nucleic acid molecule complex to be internalized by the target cell. In certain embodiments the vector in this method further includes one or more of a targeting ligand, nuclear localization sequence and endosome disrupting moiety.

### DETAILED DESCRIPTION

The present invention relates to a genetically engineered non-viral vector for delivering a nucleic acid such as a therapeutic gene to a target cell. The vectors are suitable for systemic administration to an animal. In the simplest embodiment the non-viral vector is a nucleic acid-binding protein-based polymer (NABP) having at least one tandem repeat of a genetically engineered cationic amino acid-containing monomer (CAACM), which confers on the NABP the ability to bind a nucleic acid that is intended for delivery to a target cell. The cationic amino acid in the CAACM is either lysine or arginine, or a combination of both. Because the NABP is genetically engineered and transcribed from a single gene, its structure and function can be precisely controlled. The non-viral vectors can also be used in any situation where it is desirable to bind nucleic acids, for example to remove nucleic acids from a suspension. The removal of nucleic acids would be achieved for example by complexation with a NABP followed by filtration or centrifugation.

The non-viral genetically engineered vectors (hereafter “the vectors”) of the present invention can be simple or complex, but all have predictable and controlled properties. Certain other preferred embodiments are directed to more complex multifunctional vectors, also transcribed from a single gene in a single transcript. All of the multifunctional vectors of the present invention have the NABP region which makes up the nucleic acid-binding moiety (NABM). In addition to the NABP, multifunctional vectors have one or more other optional protein moieties that introduce various specific functionalities. Such other moieties, which are discussed in detail below, include the following: a targeting ligand that targets specific cell types (e.g., receptor ligands), an endosome disrupting moiety (EDM) that disrupts endosomes (e.g., histidine or fusogenic peptides), and localization signals that traffic the nucleic acid cargo to specific sub-cellular compartments (e.g., nuclear localization signals (NLS)). Because the recombinant multi-functional vectors are all transcribed from a single gene, structure and function can be precisely correlated. Consistent production of the vectors permits increased and predictable transfection efficiency and safety. Additional advantages such as cost-effective large-scale manufacturing, purity, homogeneity, and biocompatibility make recombinant polymer vectors preferable to conventional non-viral gene vectors.

The vectors of this invention can be used in gene therapy for systemic delivery of nucleic acids to a target cell, for example a therapeutic gene to treat or prevent diseases in an animal, including a human. They can also be administered locally in situ, or used in vitro for gene transfer. While the vectors are especially desirable for gene therapy in complex organisms, they can also be used for nucleic acid delivery to unicellular animals, prokaryotes or eukaryotes, and plant cells. Systemic administration of the recombinant vector/nucleic acid complexes can be accomplished by known routes, including via intramuscular injection, intravenous administration, and intraperitoneal administration. The vector gene complexes of can also be administered as an aerosol by inhalation or other methods of administration.

Another invention is directed to genetically engineered polylysine or polyarginine, or copolymers thereof for use as vectors to deliver nucleic acids to target cells. These vectors, like the others described herein, can be engineered to be multifunctional vectors with, for example, endosome disrupting moieties, NLS and targeting ligands.

Advances in recombinant DNA technology permit the genetic engineering of large molecular weight polymers containing repeating blocks of amino acids with precise composition, sequence and length. [21-23], the entire contents of which are hereby incorporated by reference as if fully set forth herein. Over the past decade scientists have genetically engineered polymers where motifs from nature (such as collagen repeats, fibronectin moieties, elastin repeats, silk units, etc.) are combined biosynthetically at the gene construct level to produce novel biomaterials with precise sequence and composition [24-26]. Another laboratory has reported making a genetically engineered vector that consists of GAL4/Invasin for gene therapy [27]. However, the GAL4/invasion motif described was not a protein polymer-based and it did not have an NABP as is disclosed in the present invention.

Application of recombinant fusion proteins for gene therapy using DNA-binding and targeting domains has also been reported in the literature [28-30]. However, the expressed fusion proteins described in the publications listed above were not in tandem repeats producing protein-based polymers. Others have described genetically engineered protein polymers that are useful for delivering biologically active substances, particularly drugs, to a localized site in vivo. [31], the entire contents of which are hereby incorporated by reference as if fully set forth herein]. However, these protein polymers are not suitable for systemic administration because of their tendency to form gels when injected into the body. Moreover, they were not designed for and do not demonstrate the ability to bind to nucleotides in such a way as to condense the DNA to a size that can be endocytosed by the target cells.

The experiments and results presented herein represent the first time genetic engineering techniques have been used to construct a nucleic acid-binding protein-based polymer (NABP) for use in delivering a nucleic acid to a target cell, where the polymer is made of at least one tandem repeat of a cationic amino acid-containing monomer (CAACM). The CAACM is a nucleic acid-binding monomer that contains lysine or arginine or a combination thereof. Lysine and arginine are cationic amino acids that are positively charged at pH 7.4, which enables them to bind negatively charged nucleic acids. In some sensitive systems such as mammalian cells, too many repeats of lysine, arginine or combinations thereof may be toxic. Therefore in one embodiment the nucleic acid-binding protein-based polymer has from about 10% to about 70% lysine, or arginine residues, or a combination thereof, preferably between about 30% to about 60%. Routine experimentation will determine how much lysine and arginine the animal or the target cell can handle.

The cationic amino acid histidine (H) is not positively charged at pH 7.4 because the pKa of histidine is about 6. However, histidine is often included in CAACM in various embodiments of the vectors, because it disrupts endosomes facilitating the release of the nucleic acid/vector complex from the endosome. Lysine (K) and/or arginine (R) are typically repeated more than once in a CAACM, and to prevent toxicity they are often separated by an amino acid that is not positively charged at pH 7.4. Examples of CAACM including histidine include (KHKHKHKHKK), (RHRHKHC), (KHKHCKK), and (KGKHGRC). “Tandem repeat” means at least one identical CAACM after another in tandem within the NABP. A tandem repeat is identified as (monomer)n, where n is 2 to 100. One example of a tandem repeat of a CAACM in an NABP is (KHKHKHKHKK)₂—(KHKHCKK). In this example, the first segment of the polymer has a single tandem repeat of the (KHKHKHKHKK) monomer SEQ ID NO. 1 (hereafter the (KH) monomer) while the CAACM (KHKHCKK) in the second segment is not repeated. There can be more than one tandem repeat of the monomer in an NABP, for example (KHKHKHKHKK)₆ or (KKKKK)₃—(KCKKH)₂.

In addition to disrupting endosomes and diluting repetitive lysine and/or arginine residues which reduces their potential toxicity, histidine also provides a source of hydrogen bonding to nucleotides that facilitates complex formation and nucleic acid condensation. As a starting point for the experiments described below, the sequence of the KH monomer (KHKHKHKHKK) was chosen arbitrarily, keeping the lysine to histidine ratio constant at 6:4. If histidine is included in the CAACM, one embodiment is directed to CAACM having from about 10% to about 70% histidine, preferably from about 20% to about 40%. It is important to note that histidine does not need to be included in the CAACM in order to be part of the NABP. For example, the NABP can be (KXKXRRXRXK)₂—HHHHHHHHHHHHH, where X is any amino acid that is not cationic at pH 7.4. The ratio of K to H in a monomer, and in the NABP having the monomer, depends on the ability of the final construct to bind the nucleic acid and exit the endosome. See for example Midoux and Monsigny [6]. Other endosome disrupting moieties such as those described below can be added to a multifunctional vector in addition to histidine in the CAACM. Others have shown that chemically synthesized copolymers of histidine and lysine markedly enhance transfection efficiency of liposomes [5]. However, the length of such motifs that can be prepared by chemical peptide synthesis is limited, making the genetically engineered NABP of the present invention preferable. Moreover, the composition and transfection efficiency of chemically synthesized polymers are not predictable.

In one embodiment, NABP are used without further modification as a non-viral vector for delivering nucleic acids including a therapeutic gene or RNA oligonucleotide to a cell such as an animal, bacterial or plant cell. This is the simplest non-viral vector of the present invention. Any repetitive amino acid sequences that include lysine, arginine or a combination thereof, that bind and condense DNA and permit decomplexation of the DNA for expression inside the target cell can be used to make the CAACM. Lysine and/or arginine can be used in any combination with or without other amino acids to make the CAACM. In some embodiments lysine and/or arginine are interspersed in the monomer among amino acids that are not cationic at pH 7.4. The vector/nucleic acid complexes of the present invention are nano-scale particles ranging from about 10 nm to about 500 nm, preferably from about 50 to about 150 nm. This area of research falls under the broadly termed “nanomedicine” research area. Certain other inventions are directed to very simple vectors that are simply genetically engineered NABP of homopolymers of lysine or arginine, or copolymers of lysine-histidine, arginine-lysine, arginine-histidine or lysine-arginine-histidine. In some embodiments, histidine is included in the polymer alternating with lysine or arginine because histidine is an endosome disrupting moiety, and its inclusion interspersed among lysine and arginine repeats reduces toxicity that may be caused by a vector having too many cationic residues.

Example 1 describes the synthesis of the gene encoding one embodiment of an NABP, specifically (KHKHKHKHKK)₆ having SEQ ID NO. 4. For convenience, the (KHKHKHKHKK) monomer is referred to hereafter as the (KH)ₙ monomer. This (KH)₆ [SEQ ID NO. 4] embodiment of an NABP contains 36 lysine residues (K) in the (KH)₆ segment that condense nucleic acids including DNA electrostatically, and 24 histidine residues (H) that disrupt endosomes thereby permitting the vector/nucleic acid complex to escape from the endosome into the cytoplasm. Another embodiment is directed to a simple genetically engineered non-viral vector made of an NABP that has the composition (KHKHKHKHKK)₆ SEQ ID NO. 4.

The composition of the monomers and the number of tandem repeats in the multimeric NABP can be varied depending on the size of the therapeutic gene or oligonucleotide being delivered and its ability to bind to the monomers, and the size of the nucleic acid/vector complex. Factors that make genetically engineered NABP suitable candidates for systemic nucleic acid delivery include their ability to bind to and protect a therapeutic gene, and the stability and size of the polymer/gene complex in the blood stream under physiologic conditions. The inclusion of disulfide bonds between short lysine clusters by including cysteine in the monomer has been reported to enhance DNA-binding and transfection [32]. This is thought to be due to the intracellular reduction of disulfide bonds in cysteine that are formed within the polymeric backbone [32]. Therefore, in some embodiments the NABP has at least one monomer that contains one or more cysteine residues, such as (KHKHKHKHKKC)₆ SEQ ID NO. 3. The monomer (KHKHKHKHKKC) is identified by SEQ ID NO. 8.

The molecular weight of the NABP forming the nuclei acid binding moiety (NABM) of the vectors is typically from about 2,000 to about 200,000 daltons. Most commercial chemically synthesized peptide based polymers are in the average 20,000 dalton range. There is no limit on the weight of the constructs. As long as they complex with DNA and condense to form a DNA/vector particle (also herein referred to as a DNA/vector complex) that is greater than about 10 nm and below 500 nm, it is acceptable. The most effective size range for the systems studied so far is from about 50 nm to about 150 nm. The NABP will be a multimer of from about 2-100 monomeric units, preferably from about 3 to about 15 monomers. These sizes permit decomplexation and release of the therapeutic gene from the polymer once the complex is inside the target cell. It is easier to engineer a polymer that has fewer monomer repeats, however since the size of the monomer units can vary widely, the number of monomer units in the NABP will vary accordingly. An individual CAACM unit for use in making the present vectors typically has from about 3 to 35 amino acids (corresponding to 9-105 base pairs). The NABP will have at least one tandem repeat of a CAACM that contains lysine and/or arginine.

Antisense Nucleic Acids as Therapeutic Genes

RNA and other nucleic acids are also negatively charged DNA and therefore bind to the vectors of the present invention. Thus therapeutic antisense or interfering RNA can be delivered to the cytoplasm of a target cell using the vectors of the present invention where the RNA inhibits the translation of targeted proteins. Vectors for delivering RNA to the cytoplasm would have for example the NABM made of the NABP, the EDM (which could be histidine interspersed with cationic amino acids in the polymer), and possibly a targeting ligand. An NLS may be used in the event that nuclear localization is required.

The specific hybridization of certain DNA or RNA oligomers with its target nucleic acid interferes with the normal function of the target nucleic acid. This modulation of function of a target nucleic acid by compounds that specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA.

Antisense-RNA and anti-sense DNA have been used therapeutically in mammals to treat various diseases. [[33-35], the entire contents of which are hereby incorporated by reference as if fully set forth herein]. Antisense oligodeoxyribonucleotides (antisense-DNA) and oligoribonucleotides (antisense-RNA) can base pair with a gene, or its transcript such as mRNA. An antisense PS-oligodeoxyribonucleotide for treatment of cytomegalovirus retinitis in AIDS patients is the first antisense oligodeoxyribonucleotide approved for human use in the US. [36, 37], the entire contents of which are hereby incorporated by reference as if fully set forth herein].

Targeting Ligands

In various preferred embodiments the vector is genetically engineered to have one or more optional specialized moieties such as targeting ligands, endosome disrupting moieties and nuclear localization sequences that make it suitable for use in systemic gene delivery in vivo. The experiments below describe the biosynthesis and in-vitro characterization of the first prototype of a genetically engineered multifunctional non-viral vector. In one embodiment, the multifunctional non-viral vector has an NABM provided by the NABP and a targeting moiety for targeted gene delivery. Any amino acid based sequence that selectively targets a particular cell can be used to facilitate the delivery of the non-viral vector/nucleic acid complex to that particular cell. Such motifs can target any cell surface receptor such as growth factor receptors (e.g., fibroblast growth factor, epidermal growth factor, etc.) or hormone receptors. Specific cell surface antigens can also be targeted using a complementary antibody. One such targeting ligand is FGF2 that comes in high and low molecular weight forms. High molecular weight FGF2 (HMW—FGF2) is a protein of 22, 22.5 or 24 Kilo Daltons that is known to also contain a nuclear localization signal Low molecular (LMW—FGF2) (17.5 KDa) does not have an NLS [25]. Thus HMW—FGF2 is a multipurpose moiety-it is a targeting ligand and provides NLS.

Others have used chemically synthesized polylysine polymers conjugated to FGF2 to transfect various cell lines with the plasmid containing the gene encoding beta-galactosidase. The cell lines successfully transfected to express beta-galactosidase are COS-1, 3T3, baby hamster kidney (BHK) and endothelial cells, which are all FGF2 target cells [7]. The route of the vector through the cell was FGF2 specific and the vector was able to pass through the endosome. The study also showed that endosome disrupting moieties such as chloroquine and 20 amino-terminal amino acid sequence of influenza virus hemagglutinin increased protein expression by 8-fold and 26-fold, respectively. Sosnowski, et al. showed that DNA-binding to polylysine (in chemically engineered polylysine-FGF2 constructs) did not interfere with the ability of FGF2 to bind to its receptor and elicit a proliferative response in the target cells. They also showed that FGF2 has the ability to bind directly to DNA presumably because of its high isoelectric point (pI −9.56). However they did not see significant transfection by using FGF2 alone as a gene-binding moiety.

A preferred embodiment of a genetically engineered multifunctional vector having a NABM provided by the NABP and a targeting ligand is (KHKHKHKHKK)₆—FGF2. Details of the synthesis and characterization of (KHKHKHKHKK)₆-low molecular weight FGF2 SEQ ID NO. 5 are presented in Example 1. The NABP of this multifunctional vector has the structure (KHKHKHKHKK)₆ or (KH)₆. The targeting moiety is low molecular weight fibroblast growth factor 2 (FGF2). FGF2 receptors are known to be abundantly expressed in a number of malignant cell lines including, lung, colon, and ovarian carcinomas. (KH)₆—FGF2 has 36 lysine residues (K) in the (KH)₆ segment, which lysine residues condense DNA, and 24 histidine residues (H) to promote endosomal escape [38-42]. In this example, the low molecular weight FGF2 is located at the C-terminus of the construct, but the vectors can be designed and engineered to place the various functional moieties where they are most effective. Because the non-viral vectors of the present invention are transcribed from a single continuous DNA transcript, systematic and precise correlation of structure and function can be achieved that represents a significant improvement over the unpredictability of non-viral polymeric vectors made using chemical synthesis. With the genetically engineered vectors there is no uncertainty as to the position and composition of the various functional moieties in the vector. Once the optimum vector composition has been identified, genetically engineered vectors assure that all vectors made will be identical, thus eliminating the unpredictability of chemically synthesized vectors and the adverse side effects of viral vectors.

Endosome Disrupting Moiety

In certain preferred embodiments, the vectors of the present invention contain a region that disrupts endosomes typically by lysing the endosome membrane. In certain cases direct gene delivery to the cytoplasm using electropration or nucleus may be desired and so there would be no need for an endosome disrupting moiety (EDM). Endosome lysis can be accomplished by using vectors having a polymer region that is rich in histidine [9]. The optimum ratio of histidine to other amino acids in the polymer varies depending on the composition of the final construct and on the specific nucleic acid or therapeutic oligonucleotide intended for delivery. If histidine is used as an endosome disrupting moiety in a CAACM, the percentage of histidine in a CAACM can range from about 10% to about 70%, preferably from about 20% to about 40%. In one embodiment histidine is alternated with lysine or arginine in the CAACM, but this is not required. Histidine is not required for making the vectors of this invention, but if used, it can be located anywhere in the polymer. An additional advantage of using histidine in the CAACM is that histidylation of polylysine, for example, has been shown to reduce the cytotoxicity often associated with polylysine substantially enhancing transfection efficiency [6]. Introduction of imidazole side chains [9] is also reported to minimize toxicity and enhance transfection, therefore certain vectors of the present invention have NABP that include amino acids with imidazole side chains.

Without being bound by theory, it is hypothesized that a proton sponge effect occurs when histidine is protonated in the endosome at endosomal pH (˜5.0). Protonation of the histidine in the polymer is thought to induce a proton pump in the endosomal membrane, which regulates the pH of the endosome. The influx of additional protons leads to a charge imbalance in the endosome, which in turn causes an influx of chloride counterions. While the charge is now balanced, an imbalance of osmotic pressure occurs between the cytoplasm and the endosome, resulting in swelling and bursting of the endosomal compartment.

In other embodiments, the vectors of the present invention include endosome membrane fusion peptides (EMFP), which are amino acid sequences that are capable of fusing with the endosome membrane at the endosome acidic pH. Fusion of the EMFP with the endosome facilitates the release of the endosome contents, including the vector/therapeutic gene complex, into the cytoplasm. EMFP are found in various viruses. For example: viruses such as influenza virus haemagglutinin have the following EMFP sequence: (GLFEALLELLESLWELLLEA). Other known EMFP sequences include: (GLFEAIEGFIENGWEGLAEALAEALEALAAGGSC), and (VYTGVYPFMWGGAYCFCDS) in Semliki Forest Virus.

Other examples of EMFP amino acid sequences are:

KALA (WEAKLAKALAKALAKHLAKALAKALKACEA) which serves both to bind DNA and disrupt endosomes [43],

LAGA (WEAALAEAEALALAEKEALALAEAELALAA),
GALA (WEAALAEALAEALAEHLAEALAEALEALAA).
(GLFEALLELLESLWELLLEA)

Another lytic peptide called H5WYG has been reported; its amino acid sequence is: Gly-Leu-Phe-His-Ale-Ile-Ala-His-Phe-Ile-His-Gly-Gly-Trp-His-Gly-Leu-Ile-His-Gly-Trp-Tyr-Gly.

Nuclear Localization Sequences

In certain preferred embodiments, the vectors of the present invention include an NLS to direct the therapeutic gene to the nucleus where it is transcribed by the target cell. Many examples of NLS are known in the art [44-46]. Any amino acid sequence that enhances the nuclear targeting of the vector/gene complex can be considered a nuclear localization signal. An example of a known NLS that can be used in the vectors of the present invention comes from the Simian Virus SV40 large tumor antigen; the NLS comprises a single short stretch of basic amino acids (PKKKRKV) or (PNKKKRK). Other examples of NLS sequences are (RLRFRKPKSKD) in Feline Immunodeficiency Virus, (RRKRQR) in Dorsal protein, (KRRR) in adenovirus adenain protein, (RKRKR) in OCT4 protein, (RQARRNRRRRWRERQRQ) in Human Immunodeficiency Virus type 1 (HIV-1), (KSKKQK) in chicken v-rel protein, (KTRKHRG) in Ribosomal L29 protein, (GKKRSKAK) in yeast histone 2b, and (PVKKRKRK) in Rac1 protein.

Some known NLS sequences are bipartite having two stretches of basic amino acids separated by a spacer, such as is illustrated below. These include (KR-11aa spacer-KKLR) in RB protein; (RKKRK-12 aa spacer-KKSK) in N1N2 protein; (KKR-11aa spacer-KRVR) in adeno-associated virus Rep68/78 protein; (KRKGDEVDGVDEVAKKKSKK) in Poly(ADP-ribose)polymerase; (KRPMNAFIVWSRDQRRK) in Human SRY protein; (RLRRDAGGRGGVYEHLLGGAPRRRK) in Mouse FGF3; and (KRPAATKKAGQAKKKKL) in Xenopus nucleoplasmin protein.

Other known NLS sequences have charged/polar residues interspersed with non-polar residues such as the NLS [MNKIPIKDLLNPQ] in the yeast homeodomain containing protein Mat-α2.

Examples of NLS that target importin Beta include: (LGDRGRGRALPGGRLGGRGRGRAPERVGGRGRGRGTRAARGSRPGPAGTM) in high molecular weight basic fibroblast growth factor, amino acids 427-455 in Regulatory Factor X Complex; (SANKVTKNKSNSSPYLNKRKGKPGPDS) in Pho4; (VHSHKKKKIRTSPTFTTPKTLRLRRQKYPRKSAPRRNKLDHY) in rpL23a protein; and (MAPSAKATAAKKAVVKGTNGKKALKVRTSATFRLPKTLKLAR) in rpL25 protein.

Examples of some of the many NLS known in the art can be found in the following references; the entire contents of which are hereby incorporated by reference as if fully set forth herein [44-46].

The total size of the non-viral vector/therapeutic DNA or RNA complex is typically from about 10 to about 500 nm. So far, the best transfection efficiency is observed with particles below 150 nm size, from about 50 to 150 nm. The size is only limited by the ability of the target cells to endocytose the complex. The addition of one or more targeting ligands, EDM and NLS affects the size of the vector accordingly. Routine experimentation will show the optimum sizes depending on the composition of the vector, the size of the therapeutic DNA/RNA and the target cells. In some cases more than one copy of a given motif may be needed to optimize its intended function.

The examples below show various embodiments of the present invention. In these examples, the amino acid sequence in parentheses is the sequence of the monomer that is multimerized to make the NABP; the subscript following it indicates how many monomers are in tandem repeats in the NABP of the final vector. All of the embodiments of the vectors below have FGF2 as the targeting ligand. The high molecular weight form the FGF2 denoted by HMW has its own nuclear localization sequence. LMW denotes low molecular weight FGF2 that does not have an internal NLS. The histidine residues (H) are endosome disrupting moieties, and the cationic lysine (K) residues bind the nucleic acid intended for delivery to the target cell.
a) SEQ ID NO. 3 (KHKHKHKHKKC)₆; no targeting, endolytic via H, reducible via C, 6 repeats, no NLS.
b) SEQ ID NO. 12 (KHKHKHKHKK)₃—(FGF2-LMW); targetable via FGF2, endolytic via H, nonreducible, 3 repeats. The sequence (KHKHKHKHKK) is referred to as the (KH) monomer for the sake of simplicity.
c) SEQ ID NO. 5 (KHKHKHKHKK)₆—(FGF2-LMW); targetable via FGF2, endolytic via H, nonreducible, 6 repeats.
d) (LMW—FGF2); targeting moiety alone. FGF2 is both a targeting moiety and a gene-binding moiety since others have shown that it binds DNA.
e) (HMW—FGF2); targeting moiety with NLS.
f) (KHKHKHKHKKC)₃—(FGF2—HMW); targetable via FGF2, endolytic via H, reducible, via C; 3 repeats. *The sequence (KHKHKHKHKKC) will be referred to as the (KHC) monomer for the sake of simplicity.
g) (KHKHKHKHKKC)₆—(FGF2—HMW); targetable via FGF2, endolytic via H, reducible, via C, 6 repeats
h) SEQ ID NO. 6 (KHKHKHKHKKC)₃—(FGF2-LMW); targetable via FGF2, endolytic via H, reducible, via C, 3 repeats
i) The monomer (KHKHKHKHKKC) identified by SEQ ID NO. 8.
j) The monomer (KHKHCKK) identified by SEQ ID No. 9.

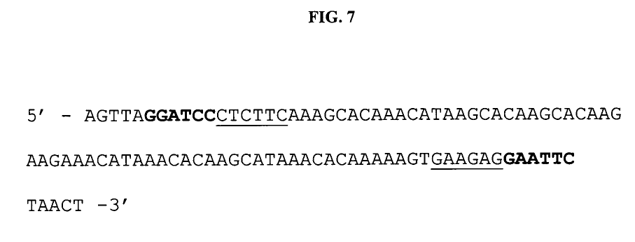

FIG. 7 shows the full oligonucleotide sequence of the sense strand of a synthetic gene used to clone the (KH) monomer SEQ ID NO. 7. The gene is designed for multimerization of a K (lysine) H (histidine) monomer with pertinent restriction sites. Starting from 5′: Bam HI recognition site (bold), Eam 1104 I recognition site (underlined), nucleotides encoding KH monomer, Eam 1104 I recognition site (underlined), Eco RI recognition site (bold). One embodiment of the invention is directed to the synthetic gene used to clone the (KH) monomer having SEQ ID NO. 7.

It is within the scope of the present invention to use gene monomers constructed solely from digestion fragments of previously constructed and sequenced monomers, in which case the final gene monomer is typically characterized by restriction digests. We have sequenced a monomer gene having 640 base pairs.

When the present vectors are mixed with therapeutic DNA, the cationic residues in the polymer in the NABM and the negatively charged nucleotides in the DNA bind with one another based on electrostatic interactions. Typically more than one vector binds with any single therapeutic gene or oligonucleotide. The number of vectors that bind to a single nucleic acid varies depending on the number of negative charges present in the nucleic acid which is related to its size, the number of positive charges available on the vector, and the length of the polymer. The vectors of the present invention themselves can be stored and stabilized in aqueous solution. The vectors that are described in the examples have been stored in phosphate or TRIS buffered solution at minus 80 degrees Celcius under which conditions they can be stored for at least 2 years. Once the vectors are at room temperature they can last about 10 hours without degrading. Experimentation will show optimal stability.

As gene transcripts for various functional motifs become commercially available, genetic engineering of the multifunctional vectors will be simplified. The genetically engineered polymer vectors of the present invention are far superior in precision to chemically synthesized polymers. Although it is preferable to engineer the entire vector, in some cases it may be expedient to use chemical means to attach various functional motifs chemically to genetically engineered vectors.

In previous studies we tested the ability of many of the commercially available poly (amino acid)s to bind, protect and deliver genes to Cos-7 cell lines (African green monkey kidney) in vitro without degradation in endosomes, without causing cytotoxicity, and with efficient transfection rates and levels of gene expression. [[47], the entire contents of which are hereby incorporated by reference as if fully set forth herein.] Random copolymers of poly ‘(Lys, Ala) 1:1], poly [Lys, Ala) 2:1], poly [(Lys, Ala) 3:1], poly [(Lys, Ser) 3:1] and poly [(Arg, Ser) 3:1] were complexed with plasmid DNA containing cDNA encoding Renilla luciferase (Rluc) at different weight per weight DNA/Polymer ratios. These polymers were commercially available and prepared by chemical means. All of these chemically synthesized polymers were able to condense DNA, but the optimum DNA/Polymer ratios varied depending on polymer structure and molecular weight. There is a wide range of DNA/polymer ratios which can be used to condense DNA though efficiency varies. The particle sizes of the DNA/polymers in these studies varied in the 100-350 nm range, which is suitable for systemic administration. The vector/nucleic acid complexes of the present invention range from 10-500 nm, preferably from about 50-150 nm. Because of the precision of genetic engineering, new amino acid based polymers can be designed to have the optimum ratio and sequence for condensation, targeting, endosomal escape and nuclear localization.

The following is a non-limiting general outline for making the genetically engineered non-viral vectors of the present invention. The details for specifically making the (KH)₆—FGF2(LMW) vector are set forth in Example 1.
1) First, is the design of an amino acid polymer (Nucleic acid-binding protein-based polymer) made up of monomers that have the desired cationic amino acid pattern for binding nucleotides,
2) make a gene sequence that has a nucleotide sequence encoding the monomer,
3) insert the sequence into an acceptor plasmid for transforming bacteria thereby making a cloning vector,
4) transform bacteria with the cloning vector,
5) grow the transformed bacteria on agar plates with selective antibiotics to isolate the colonies bearing the cloning vector,
6) pick a colony and grow it in culture media for a length of time for cloning vector propagation,
7) extract the plasmids from the transformed bacteria from step 6,
8) cut the plasmids with restriction enzymes to cut out the monomers,
9) run on agarose gel to purify the monomer genes,
10) do a PCR to amplify the genes for the monomers with proper restriction sites,
11) multimerize the amplified monomers by self ligation to make concatemers,
12) ligate the concatemers into an expression vector,
13) transform the expression vector into bacteria and plate on agar plates with selective antibiotic to isolate the colonies baring the expression vector,
14) screen the colonies to identify the bacterial colonies that express the monomer gene or multimers of the monomer,
15) picking the colony of interest and grow in culture media,
16) isolate plasmids having the desired concatemer genes from the bacteria in step 14,
17) perform PCR to amplify the multimer gene and introduce new restriction sites suitable for fusion with other genes such as the gene for targeting ligands such as FGF2 (or other moieties, EDM or NLS),
18) amplify the gene encoding FGF2 (or other desired motifs) and introduce proper restriction sites,
19) digest the FGF2 gene and cloning vector with proper restriction enzymes,
20) ligate FGF2 gene into the cloning vector,
21) digest cloning/expression vector containing FGF2 gene with proper restriction enzymes for fusion with amplified gene in step 16,
22) ligate multimer gene segments from step 16 into digested cloning/expression vector from step 20,
23) transform E. coli with cloning/expression vector and plate on agar with proper antibiotic,
24) pick a colony from agar plates and grow it in culture media,
25) isolate the cloning/expression vector and verify the presence of the correct gene by DNA sequencing,
26) transform an E. coli expression host with the verified plasmids and induce the cells by IPTG, Arabinose or heat,
27) harvest the cells, then lyse them,
28) load the lysate onto purification column for protein purification,
29) collect the purified protein which is the protein based (amino acid) polymer of the desired sequence as a NABM plus FGF2 or other motifs that have been added.

The skilled artisan is aware that the steps described above may or may not be taken and/or executed in the order given. Modifications to routine and well-known methods in the field of molecular biology may be made to the steps described above within the scope of the present invention. Example 2 has details of hypothetical methods for the synthesis and characterization of genes encoding (KHC)ₙ—FGF2.

Ferrari, et al. U.S. Pat. No. 5,830,713 describes methods for preparing synthetic repetitive DNA. Prior to the discoveries in Ferrari, et al. high-molecular-weight polymers containing repeating sequences of amino acids had been difficult to produce by biochemical means. The genes necessary for producing large protein based polymers containing repeating units of amino acids were unstable and often underwent intermolecular recombination causing deletions of repeating units in the gene. U.S. Pat. No. 5,830,713, the entire contents of which are hereby incorporated by reference as if fully set forth herein). However, the Ferrari patent did not describe making polymers of repetitive cationic amino acid monomers or their use as vectors for gene therapy.

The methods for producing of genetically engineered NABP according to the present invention involve preparing a double stranded (ds) DNA “monomer” having the desired DNA sequence using the seamless cloning technique described in Example 1. Seamless cloning is a relatively new technique described in [[48], the entire contents of which are hereby incorporated by reference as if fully set forth herein]. The presence of small “trash” residues encoding amino acids such as glycine or alanine between monomers in the final concatemer can be tolerated. Routine experimentation will show the tolerance of trash amino acids for each different therapeutic gene. The dsDNA monomer is an extended segment of DNA principally encoding DNA-binding and endosome disrupting cationic amino acid repeating units. The final product encoding the polymer will be a multimer of from about 2-100 monomeric units, preferably from about 3 to about 15. These sizes permit decomplexation and release of the therapeutic gene from the polymer. Gene monomers were self-ligated to produce multimer gene segments or concatemers (which are DNA segment composed of repeated sequences linked end to end), which were then cloned. As is described below, genes for the other motifs to be included in the final vector are also each cloned. Once each gene segment for the various functional motifs of the vector is cloned, the next step is to harvest the genes and connect them to the concatemerized NABP gene segment. The entire gene for the combined polymer NABP, targeting ligand, EDM and/or NLS is then cloned and expressed to make the non-viral vectors for use in gene therapy. Cloning strategies may vary depending on the type of the desired amino acid sequence.

In one embodiment, the genes for the NABP gene-binding moiety comprise at least one tandem repeat of the monomers of DNA, each monomer encoding the same amino acid sequence, for example the monomer (KH)₂. In other embodiments, all or a part of two or more different monomers encoding different amino acid repeating units may be joined together to form a new monomer such as a block copolymer that is then concatemerized into the gene to make the NABP nucleic acid-binding moiety.

Within a monomer, dsDNA encoding the same amino acid monomer repeating unit may involve two or more different nucleotide sequences, relying on the codon redundancy to achieve the same amino acid sequence. For example AAA and AAG codons both encode for lysine. E. coli inserts AAA almost three times more often than AAG during transcription. Basically, the likelihood of using AAA vs AAG in E. coli to encode lysine is 0.719 to 0.281. It is desirable to optimize code degeneracy in designing the synthetic genes encoding the polymer.

The actual placement of NABP in the final genetically engineered vectors of the present invention where the vector has one or more additional functional motifs can vary. For example the NABP domain and the nuclear localization sequence can be placed either at N-terminal or C-terminal end of the targeting ligand. The proper position of the NLS, targeting ligand, EDM or polymer will be determined experimentally and optimized to maximize transfection efficiency depending on the therapeutic gene and the target cells. The final construct can be: NABP—NLS—FGF2 or NLS—FGF2—NABP and so forth. In Example 1, the polymer is located at the N-terminal of the targeting motif (FGF2).

Example 2 describes materials and methods proposed for making vectors that have cysteine in the monomer repeats.

Results

(KH)₆-LMW—FGF2 Cloning, Expression and Identification

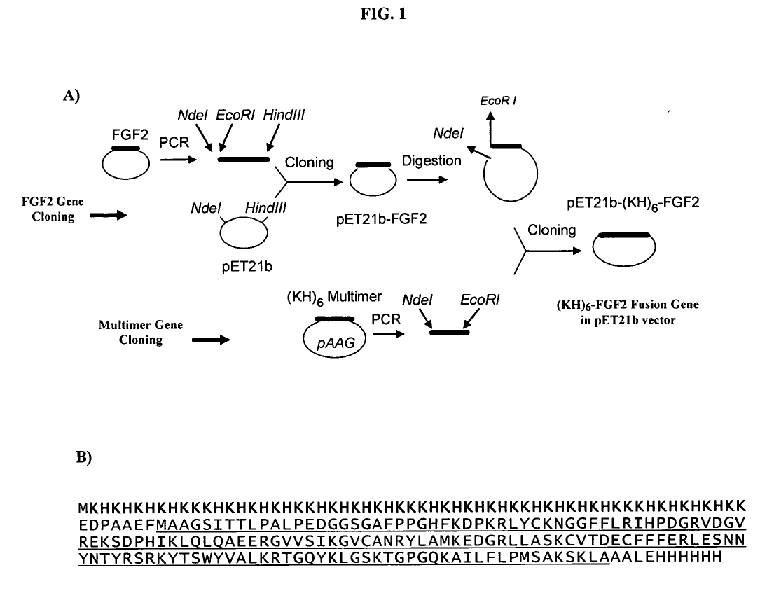

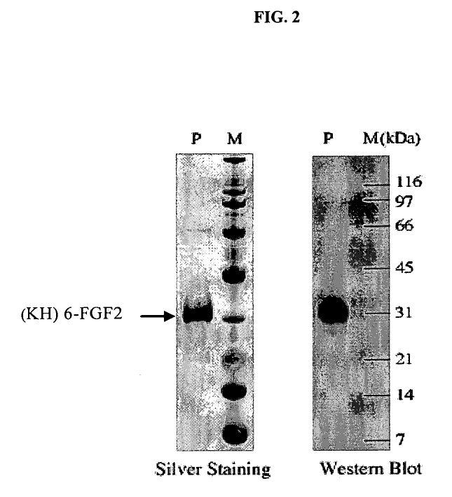

Using the cloning strategy shown in FIG. 1A, the (KH)₆—FGF2 protein polymer was cloned and expressed. The fidelity of both sense and antisense strands was confirmed by DNA sequencing, yielding the amino acid sequence in FIG. 1B. Under the stated conditions about 900 μg of vector was purified from a 1 liter culture. The purity and expression of the protein (vector) was determined by SDS-PAGE (FIG. 2A) and western blot analysis using rabbit Anti-LMW—FGF2 (data not shown) and mouse Anti-6×His (FIG. 2B). The molecular weight of (KH)₆-LMW—FGF2 was determined by MALDI-TOF to be 27,402. The results of the amino acid content analysis agreed with the expected amino acid compositions. Details are set forth in Example 1.

Plasmid DNA Condensation and Particle Size Analysis

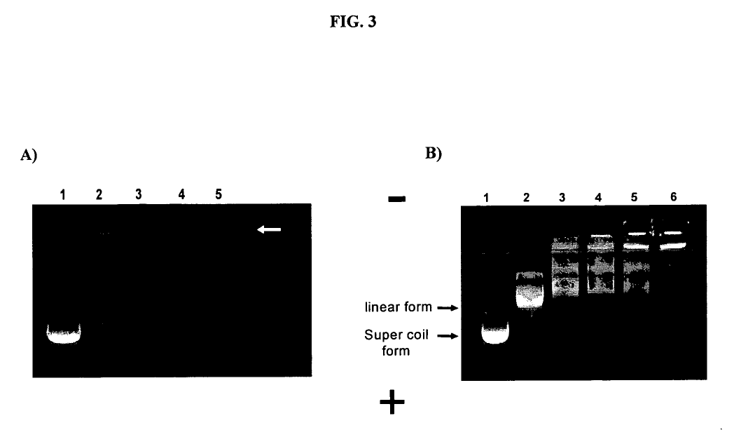

The ability of the (KH)₆-LMW—FGF2 vector to condense model plasmid DNA (PEGFP) was examined in the presence of fetal bovine serum 10% (v/v). Gel retardation assays indicate that the vector does interact with DNA retarding its migration in a dose-dependent manner (FIG. 3). The pDNA net negative charges were fully neutralized by vector at a 1:100 mole/mole ratio in 5 mM PBS and the size of the pDNA/vector complexes at this ratio was determined to be 231±15 nm by photon correlation spectroscopy. The size of the complexes at ratios below 1:100 and above 1:140 were measured to be >500 nm and >800 nm, respectively. One embodiment of the invention is directed to DNA/vector complexes that have a size of less than 400 nm, which corresponds to a ratio of from about 1:100 mole/mole ratio in 5 mM PBS to about 1:140. (KH)₅ failed to form stable nano-size complexes with pDNA in 5 mM PBS but was able to condense pDNA into 225±12 nm particles in water. These results show that the (KH)₅ does not bind to DNA efficiently, which in turn results in lower transfection efficiency. When the size of the polymer was increased to (KH)₆, FGF2 was able to bind and condense DNA efficiently.

Mitogenic Activity and Toxicity of (KH)₆—FGF2

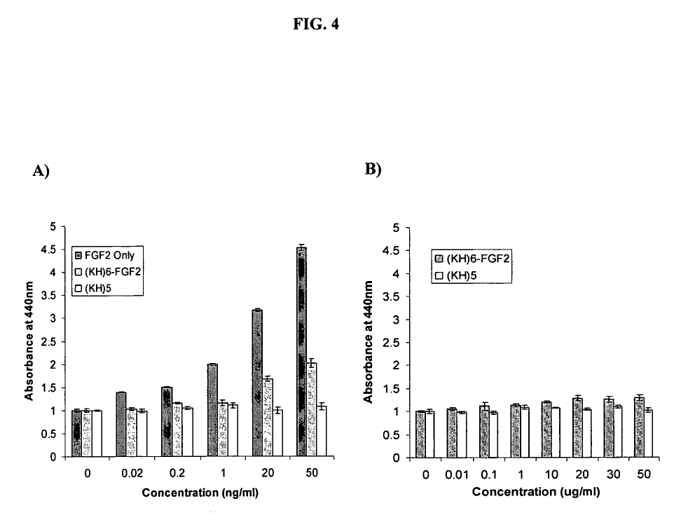

The bioactivity of the LMW—FGF2 segment of (KH)₆-LMW—FGF2 was evaluated and compared with native LMW—FGF2 and (KH)₅ with WST-1 cell proliferation assay in NIH 3T3 fibroblasts, known to express the FGFR. The LMW—FGF2 motif present in (KH)₆-LMW—FGF2 was shown to be active in terms of inducing cell proliferation in fibroblasts when they were exposed to concentrations of vector that mimicked physiological FGF2 levels (FIG. 4A). The toxicity of (KH)₆-LMW—FGF2 in NIH 3T3 cells (grown in serum free media or complete growth media) exposed to super-physiological doses of vector ranging from 10 to 50,000 ng/ml was also determined (FIG. 4B). It was observed that (KH)₆-LMW—FGF2 did not have any deleterious effect on the cell proliferation rate regardless of the dose or growth media used. (KH)₅ had no toxic or mitogenic activity in the range examined.

In-vitro Cell Transfection Is Mediated by (KH)₆—FGF2

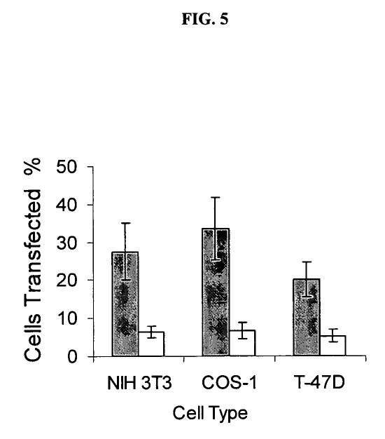

To evaluate transfection efficiency, the pEGFP plasmid, encoding green fluorescent protein (GFP) was condensed with (KH)₆-LMW—FGF2 and used as a reporter to monitor the percentage of transfected cells in three cell types expressing FGFR: NIH 3T3, COS-1, and T-47D cells, in the presence and absence of serum. Transfection was observed in all cell lines, regardless of whether serum was present, though the percentage of transfected cells was significantly higher in the absence of serum. Transfection efficiencies ranging from minimum of 15% (T-47D cells) to a maximum of 41% (COS-1 cells) were observed in the absence of serum. Transfection efficiency was 4 to 10% in the presence of serum (FIG. 5). For lipofectamine, transfection efficiency ranged from 40-65% in serum free media and from 30-51% in the presence of serum. Lipofectamine is a standard transfection agent that was used for comparison purposes to guide the systematic modification of the structure, which is now possible due to genetic engineering techniques, to correlate structure with function. The transfection efficiency for (KH)₅ ranged from 1 to 2% in the presence and absence of serum. To evaluate whether specific uptake was occurring through FGFR, we conducted transfection experiments on NIH 3T3 cells in the presence of 1000 ng/ml free FGF2. Under these conditions, we observed a reduction in transfection efficiency of approximately 85% (from 28±9% to 4±2%) showing that uptake was dependent on FGF receptors. The transfection efficiency in the blood which contains serum is expected to increase with a higher number of nucleic acid-binding monomers in the nucleic acid-binding polymer portion of the vectors even in the presence of blood serum. Overall, higher transfection was observed in SFM. Lower transfection efficiency in the presence of serum could be the result of serum protein and salt interference with the DNA/vector complexes.

To summarize the studies presented herein, vectors high in lysine and histidine content and therefore highly basic were expressed in E. Coli cells induced to divide under stringent conditions to minimize potentially adverse effects of expressed protein on growth, which in turn could increase the frequency of mutations. The purified polymer was analyzed by SDS-PAGE and the observed molecular weight was approximately 30,000 Daltons which is greater than the expected 27,313 Daltons. This discrepancy is probably due to the highly cationic nature of the vector, which retards migration in SDS-PAGE. The molecular weight of the protein (vector) was further analyzed by mass spectroscopy (MALDI-TOF) to determine the exact molecular weight of (KH)₆-LMW—FGF2. The slight difference observed between the expected molecular weight (27,313) and measured by MALDI-TOF (27,402) could be related to the peak width at half height calculations during the molecular weight measurements by MALDI-TOF. Amino acid content analysis provided more information regarding the identity of the expressed vector which showed good agreement between the expected and observed percentage of each amino acid in the structure of the expressed protein vector. These results demonstrated the first successful genetic engineering of an amino acid polymer-based vector for gene delivery, the (KH)₆—FGF2 vector.

Cationic amino acids, such as lysine, condense DNA by neutralizing the negative charges of phosphate groups and, therefore, decreasing the columbic repulsions between DNA phosphates and promoting hydrophobic interactions at the complexed sites. Stability of condensed DNA in physiological conditions is one of the major hurdles for its use in gene therapy. Even though DNA particles are shown to be stable in salt-free environments, in the presence of physiological saline or serum they often fail to remain stable. We prepared complexes of DNA and amino acid polymer-based vector prepared in the absence of serum to determine the ratio at which DNA is fully condensed. By adding (KH)₆-LMW—FGF2 to pDNA in escalating concentrations, the negative charges on pDNA were neutralized and reduced electrophoretic mobility was observed (FIG. 3A). Subsequently, serum was added to the complexes and the electrophoretic mobility of the complexes was examined (FIG. 3B).

FIG. 3B shows the migration of naked pDNA in the presence and absence of serum (Lane 1 and 2). The retardation of DNA migration on agarose gel as shown in FIG. 3B (Lane 2) could be the result of binding with serum proteins as well as linearization of DNA by serum endonucleases. It was also observed that (KH)₆-LMW—FGF2 and pDNA at 1:100 mole/mole ratio (FIG. 3, Lane 6) remained complexed in the presence of serum proteins and salts.

The DNA encoding pEGFP has 4,731 base pairs which corresponds to about 9,462 negative charges, while (KH)₆-LMW—FGF2 has only 60 cationic amino acids (49 lys+11 Arg). There are 49 lysine residues in (KH)6+FGF2, and 11 Arg in FGF2,which corresponds to about 60 positive charges. Full condensation of 1 mole pEGFP was accomplished with 100 mole (KH)₆-LMW—FGF2 as determined by gel retardation assay; every 1.6 negative charges (N) on the DNA was neutralized by 1 positive charge (P) on the polymer NABM region of the vector. Condensation of every 1.6 negative charges with 1 positive charge may be due to the active contribution of histidine residues in DNA condensation via hydrogen bond formation. Considering the total number of histidine and lysine residues in DNA condensation calculations, the N/P ratio was determined to be about 1:1. Therefore, it is plausible that not only cationic residues contributed to DNA condensation; residues such as histidine which can form a hydrogen bond with DNA may also have played a role.

The size of the vector/DNA complexes plays an important role in DNA internalization by the target cells. Although there is no consensus on whether particles with smaller size are more effective than larger ones, complexes should at least be small enough to be endocytosed. The average particle size of the vector—DNA complexes, at a ratio of one DNA molecule per 100 vector molecules, was 231±15 nm. The sizes of the complexes at ratios below 1:100 were above 500 nm which could be the result of partial DNA condensation. At DNA:vector ratios above 1:140 the size of the particles dramatically increased (i.e., >800 nm). This could be the result of particle aggregation caused by the hydrophobic interactions between FGF2 amino acid residues. Thus, one embodiment of the invention is directed to vector—DNA complexes with a size from about 10 nm to about 500 nm, preferably below 150 nm. Others have reported that high molecular weight cationic polymers such as poly-lysine are moderately toxic in mammalian cells in culture [49].

We studied the toxicity of the (KH)₆-LMW—FGF2 vector under two conditions; a) in serum free media with no protein other than transferrin and insulin, and b) in complete growth medium supplemented with serum. Since the transfection studies were performed under both serum-free and serum-containing conditions, this study provided insight as to whether the vector had any toxic effect on cells at high concentrations that could negatively impact transfection efficiency. It was observed that (KH)₆-LMW—FGF2 did not have any deleterious effect at concentrations up to 50,000 ng/ml on the cell proliferation rate regardless of the dose of serum (10% serum, 90% culture media v/v) or growth media.

Having shown that the (KH)₆-LMW—FGF2 is biologically active and can bind pDNA, the ability of the vector to deliver pDNA into cells expressing FGFR was next evaluated in NIH 3T3, COS-1 and T-47D cells known to express FGFR [7, 50]. Cell transfection was conducted in serum free media (SFM) and in growth media supplemented with serum (complete growth media). In complete growth media proteins are present that might interact with (KH)₆-LMW—FGF2/pEGFP complexes. The presence of growth factors in serum can also potentially inhibit the receptor mediated endocytosis of these complexes. By contrast, SFM does not have any growth factors that compete with (KH)₆-LMW—FGF2 and there are only two proteins (i.e., insulin and transferrin) present in the media. As expected, a higher percentage of cells were transfected in SFM than in the presence of complete growth media (FIG. 5).

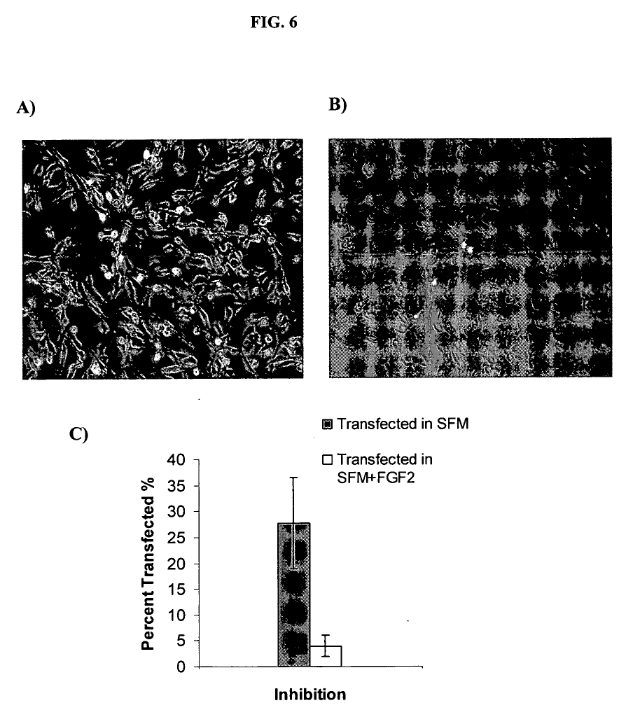

To determine whether the (KH)₆-LMW—FGF2/pEGFP complex is specifically delivered to cells via FGF2 receptor-mediated endocytosis, a competitive inhibition study in SFM was performed in the presence of 1000 ng/ml free FGF2 as a competitor. Under these conditions, a reduction in transfection efficiency of approximately 85% (from 28±9% to 4±2%) was observed (FIG. 6). The lack of full inhibition could be the result of particle uptake via non-specific endocytosis. These results show that the vector/DNA complexes are delivered specifically through the FGF receptors. The ability of the LMW—FGF2 motif of the vector (the targeting moiety) to bind to FGF receptor enables the (KH)₆—FGF2 vectors to target cells such as cancer cells that express FGFR.

Proteins are known to degrade rapidly or lose their activity when their conformations are altered by mutations, incorporation of amino acid analogs, denaturation or premature chain terminations. These modifications may prevent proper folding or disrupt protein structure, which can make the resulting aberrant protein prone to degradation or inactive. It is important that the various domains in the multi-motif fusion proteins (the vectors) of the present invention not interfere with one another and rendering any domain inactive. The (KH)₆-LMW—FGF2 fusion polymer-protein embodiment of the present invention has two moieties, (KH)₆ (the NABM) and FGF2 (the targeting moiety). It is important that the vector has the ability to both condense DNA and bind FGFR. To test the biological activity of the LMW—FGF2 moiety, NIH 3T3 cells that express FGFR were incubated with (KH)₆-LMW—FGF2 at concentrations close to physiologic FGF2 concentration, which is in the range of about 0.1-10 ng/ml. The results showed that (KH)₆-LMW—FGF2 significantly enhanced cell proliferation, indicating that the addition of a lysine-histidine domain to the LMW—FGF2 did not render the LMW—FGF2 domain inactive. Thus we showed that the (KH)₆-LMW—FGF2 vector is able to mediate gene transfer in various cell lines in an efficient and nontoxic manner.

### EXAMPLES

### Example 1

### Cloning of the (KH)₆-LMW—FGF2 Vector

A. Cloning Gene Monomer Segments (the Gene Binding Motif)

The methods herein describe the stable cloning of the gene monomer segments encoding the lysine and histidine repeat for further multimerization. The oligonucleotides encoding lysine-histidine (KH) monomers were designed to maximize the use of preferred codons in E. coli, while minimizing the codon repetition of the monomer gene. Restriction sites used for cloning into the cloning vector (pZero-2 by Invitrogen, CA, USA) and the expression vector (pAAG) were also included (FIG. 7). In brief, oligonucleotides encoding the monomer with BamHI and EcoRI (Shown in Bold), 5′-AGTTAGGATCCCTCTTCAAAGCACAAACATAAGCACAAGCACAAGAAGAAACA TAAACACAAGCATAAACACAAAAAGTGAAGAGGAATTCTAACT-3+.

Oligonucleotides encoding the monomer were first annealed in STE buffer (10 mM TRIS, pH 8.0, 50 mM NaCl, 1 mM EDTA). The double-stranded oligonucleotides were desalted with a size exclusion column and digested with BamHI and EcoRI restriction enzymes. Simultaneously, the pZero-2 vector was digested with the same enzymes. After removal of the enzymes by phenol chloroform extraction, and concentration of the DNAs by ethanol precipitation, the monomer DNA and pZero-2 vector were ligated overnight, at 16° C., with T4 DNA ligase. The ligation mixture was transformed into E. coli TOP10 cells, which were subsequently plated on Luria Broth (LB) agar containing the selective antibiotic kanamycin (37.5 μg/ml). Colonies were screened by PCR colony screening, and the insertion of the desired monomer(s) was verified by plasmid minipreparation and restriction digestion of pZero-2 with BamHI and EcoRI. The colonies expressing the desired monomer were selected. The program was optimized for PCR colony screening of the monomers.

The DNA sequence of the monomer gene was confirmed. The monomer gene segment encoding 10 repeats of lysine and histidine (total 20 amino acids) was stably cloned and successfully obtained. The number of histidine residues is 40% of the total lysine-histidine residues. This amount (40%) was chosen based on previous studies demonstrating that a chemically synthesized polylysine where 38±5% of the gamma-amino groups of lysine were substituted with histidyl residues, mediated transfection several orders of magnitude greater than polylysine, polylysine+chloroquine, or polylysine+E5CA (an endosomolytic peptide) [6].

As an alternative strategy to that described above, these small multimers can be fused to a GST or NUS tag which exist in some commercially available pET cloning/expression vectors. These tags can increase the solubility and the size of the expressed vector, hence, preserving them from the deleterious effects of proteases. In addition the cationic nature of the constructs can be toxic to the host cells. Alternative organisms such as yeast can be used for this purpose.

The expression vector for the polymer of amino acid monomers that makes up the gene-binding moiety is characterized by having an origin of replication which is functional in an appropriate expression host, usually for episomal maintenance, and a marker for selection.

As general background the expression vector for the protein based polymer also has a promoter which is functional in the expression host. Various promoters can be used, which provide for a high level of transcription, either inducible or constitutive transcription. Illustrative promoters include beta.-lactamase, beta.-galactosidase, lambda.P.sub.L or .lambda.P.sub.R promoters, trpE promoter, trp-lac promoter, T7 promoter (particularly genes 9 and 10 ), cI.sup.ts, etc. The multimer gene and the linearized vector may be combined under hybridizing, usually including ligating, conditions. Where the multimer gene does not have an initiation codon, such a codon can be added. More conveniently, the multimer gene may be inserted into a coding sequence present in the vector, under the transcriptional control of a promoter. Instead of seamless cloning, other methods known in the art can be used as long as “trash” amino acids do not interfere with the functioning of the polymer.

Instead of a vector, DNA constructs may be employed for transformation of the expression host, with integration of the construct into the genome of the expression host. The construct will differ from the vector primarily by lacking an origin which provides for episomal maintenance. Thus, the construct will provide at least transcriptional and translational initiation and termination regions, the gene encoding the protein based polymer between the initiation and termination regions and under their regulatory control, a marker for selection as described above, and other functional sequences, such as homologous sequences for integration into the host genome, sequences for priming for the polymerase chain reaction, restriction sites, and the like.

B. Preparation of DNA Encoding Lysine-Histidine Concatemers

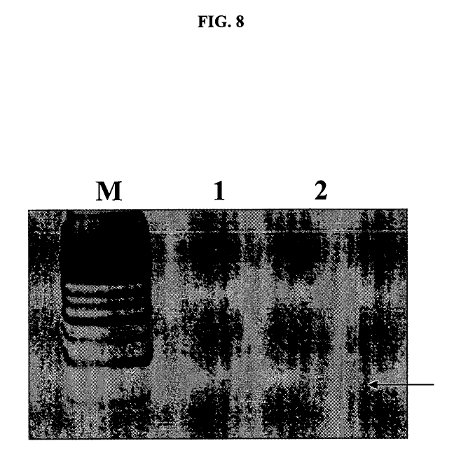

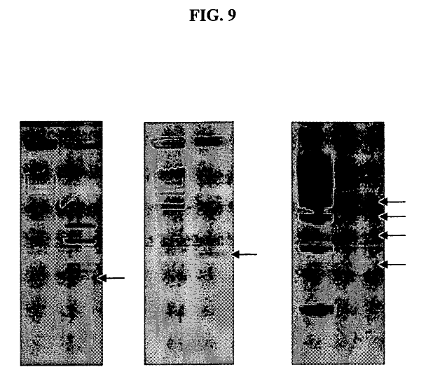

Gene monomers were self-ligated to produce multimer gene segments or concatemers. Concatemers were produced by first performing PCR directly on an E. coli TOP10 colony containing the (KH)₂ monomer gene. Ten 20 μl PCR reactions were performed and combined to produce enough monomer DNA (˜1 μg) for one cloning effort. The monomers were purified with a Qiaquick PCR purification kit and digested with the Eam 1104 I restriction enzyme. After digestion, the monomers were phenol-chloroform extracted, ethanol precipitated, resolubilized in sterile water, and run on a 20% polyacrylamide gel. Four bands were observed (FIG. 8), corresponding to undigested PCR product, two PCR products digested on one side and digested monomer on both sides. The band corresponding to the monomer was excised from the gel and purified according to standard methods. After purification, the recovered monomer was self-ligated with T4 DNA ligase for one hour at room temperature, to form concatemers. Gel electrophoresis of the ligation mixture on a 15% polyacrylamide gel showed a series of concatemers that result from self-ligation of the monomers (FIG. 9).

C. Preparation of the pAAG Vector.

A custom-designed vector was made for cloning of the KH concatemers. This vector was named pAAG, because of its engineered 5′ three-base AAG overhang. The basis for this vector was the pET-19b expression vector, which contains a histidine tag at the N-terminal end of the cloning site, followed by an enterokinase cleavage site. Since the vector does not contain any restriction enzyme recognition sites that could be used to seamlessly clone the KH DNA concatemers, customized sites were engineered. This was done by inserting two Sap I restriction endonuclease recognition sites within the cloning site on the vector. Similar to Eam 1104 I, Sap I is a type IIs restriction enzyme that removes its own recognition site. By inserting the appropriate nucleotide sequence downstream from the cleavage site, 5′ three-base overhangs were engineered that are compatible with the KH concatemers.

D. Cloning of the Concatemer DNA

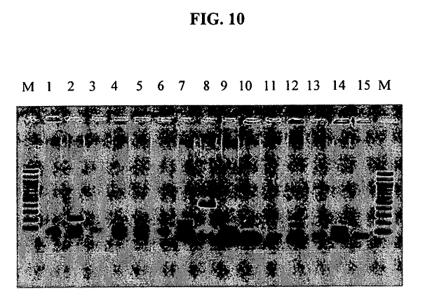

After preparation of the purified concatemer and vector, the two were ligated with T4 DNA ligase. The reaction was allowed to proceed for 16 hours, at 16° C. The ligation mixture was used to transform E. coli TOP10, which were plated on LB agar containing 50 μg/ml carbenicillin. Colonies were screened by PCR colony screening, to determine the approximate size of the insert (FIG. 10). The size and sequence of the insert was verified by triple DNA sequencing. Results showed stable cloning of lys-his repeat units. Sequences in lane 2 corresponding to 30 repeats of lysine-histidine (total 60 amino acids) and lane 8 corresponding to 60 repeats of lysine-histidine (total 120 amino acids) were chosen for ligation with LMW—FGF2 gene and further expression.

E. Cloning and Expression of Low Molecular Weight FGF2

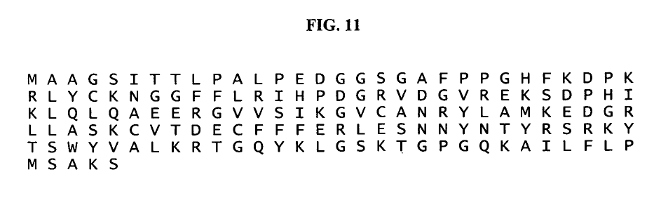

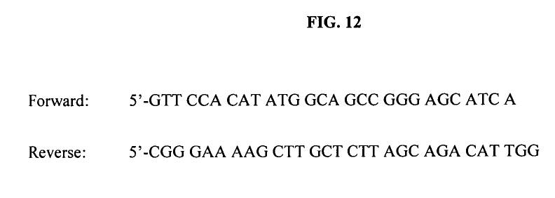

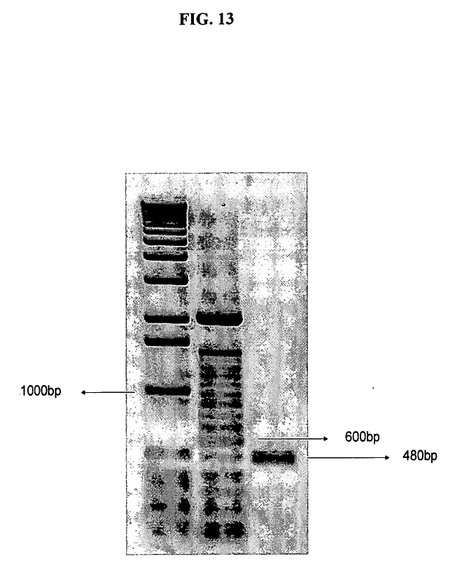

Once the Lys-His gene multimers were stably cloned and isolated, the next step was to separately clone and express a prototype targeting moiety, namely LMW—FGF2 (FIG. 11). The plasmid cDNA containing the human LMW—FGF2 gene (generously donated by Dr. Patricia Dell'Era, Dept. Biomedical Sciences & Biotechnology, Unit of General Pathology and Immunology, Brescia, Italy) were transformed into DH5α subcloning efficiency E. coli cells and sufficient plasmid DNA was obtained following Qiagen™ mini-prep purification protocol. Using PCR and proper primers, the LMW—FGF2 gene was amplified from the plasmids as shown in FIG. 12 using the following primers: Forward (NdeI and EcoRI sites underlined) GTTCCACATATGGGGGAATTCATGGCAGCCGGGAGCATCA; Reverse (HindIII underlined): CGGGAAAAGCTTGCTCTTAGCAGACATTGG. The amplified gene was double digested with NdeI (New England Biolabs, MA, USA) and HindIII (New England Biolabs, MA, USA) and purified by agarose gel electrophoresis. The LMW—FGF2 gene was then cloned into a pET21b (Novagen, CA, USA) vector that was previously double digested with NdeI and HindIII. A gene encoding (KH)₆, which corresponds to 30 lysine-histidine repeats (total 60 lysine-histidine) was then amplified from the pAAG vector by PCR using the primers: Forward (NdeI site underlined): GACGACGACAAGCATATGAAGCAC; Reverse (EcoRI site underlined): CGGGTTGAATTCAGCAGCCGGATCCTCCTTTTT. The amplified (KH)₆ gene and pET21b-LMW—FGF2 were double digested with NdeI and EcoRI.(1.5 hours, 37° C.) in total volume of 20 μl. The vector was treated with 1 ml of CIP (Calf Intestinal Alkaline Phosphatase) to prevent the re-ligation of the vector. The digested gene and vector were both loaded on agarose gel and purified using Qiagen™ Gel Extraction kit and protocol. The amplified genes were then ligated with Quick T4 DNA Ligase (20 minutes, 25° C.) to form the vector pET21b-(KH)₆-LMW—FGF2. The PCR product was loaded onto agarose gel and electrophoresed to confirm the size of the amplified gene (FIG. 13). The band related to the LMW—FGF2 gene was cut out and purified using Qiagen™ Gel Extraction Kit and protocol.

The amplified vector was transformed into E.coli NovaBlue BL21(DE3) (lon⁻, ompT) (Novagen, CA, USA) and was subsequently plated on LB agar containing carbenicillin (100 μg/ml). To confirm the insertion of LMW—FGF2 gene into pET21b vector, pET21b-LMW—FGF2 was transformed into DH5α and sufficient amount of plasmid (pET21b-LMW—FGF2 ) was isolated. The pET21b-LMW—FGF2 vector was double digested with NdeI and HindIII and loaded on agarose gel to confirm the insertion of the LMW—FGF2 gene. Also, pET21b-LMW—FGF2 was sequenced and the insertion of LMW—FGF2 gene into pET21b vector was reconfirmed.

F. LMW—FGF2 Gene Expression.

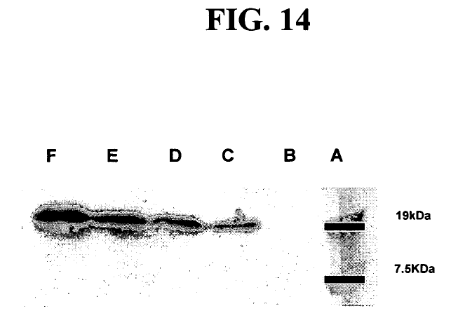

The LMW—FGF2 was expressed to serve as control (targeting moiety without carrier or NLS). pET21b-LMW—FGF2 vector was transformed into BL21 (DE3) expression host cells. Colonies were selected to inoculate a 50 ml LB media culture. The cells were induced with IPTG and grown for 4 hours. 500 μl samples were taken at every hour for 4 hours (i.e., 4 samples). The cells were harvested and lysed with lysis buffer. The soluble fraction was removed and analyzed by western blot using Anti-FGF2 as the primary antibody (FIG. 14), which binds to all isoforms of the FGF2 (Low and high). It is not specific to LMW—FGF2 or HMW—FGF2.The results show the expression of LMW—FGF2 with increasing in band intensity as time progresses. BL21(DE3) cells were used to express LMW—FGF2 in 1 liter LB media and the protein was purified using Ni-column chromatography. The expressed protein will be studied for its proliferation activity on NIH3T3 or T-47D cells over-expressing FGF2 receptors.

G. Cloning and Expression of (KH)₆-LMW—FGF2

Once the stable cloning of each gene segment was demonstrated, the next step was to connect the Lys-His multimer gene segment with LMW—FGF2 gene segment and clone and express a prototype polymer. The gene for the gene-binding and endolytic moieties can be similarly connected to genes for the other moieties as was described above: the targeting ligand, and NLS. The LMW—FGF2 gene was amplified by PCR to produce (KH)-LMW—FGF2. The LMW—FGF2 gene has proper restriction sites to be fused with the (KH)₆ gene. The KH-LMW—FGF2 gene was first digested with NdeI and HindIII and cloned into pET21b to make pET21b-(KH)-LMW—FGF2. The (KH)₆ gene, which corresponds to 60 lysine-histidine repeats, was amplified from the pAAG vector by PCR. The (KH) monomer is actually (KHKHKHKHKK). The (KH)₆ gene and pET21b-KH-LMW—FGF2 were digested with NdeI and EcoRI separately in a 20 μl reaction mixture (1.5 hours, 37° C.) and loaded onto agarose gel and purified using Qiagen™ Gel Extraction kit and protocol. (KH)₆ was cloned into pET21b-KH-LMW—FGF2 using Quick T4 DNA Ligase (20 min, 25° C.) and transformed into DH5α cells. A colony was selected and grown overnight to obtain enough plasmids using the Qiagen mini-prep kit and protocol. The cloned vectors were sequenced and insertion of both lysine-histidine repeats and LMW—FGF2 into pET21b was confirmed.

H. Expression and Purification of (KH)₆-LMW—FGF2

The pET21b-(KH)₆-LMW—FGF2 vector was transformed into BL21 (DE3) host and expressed. 500 microliter samples were taken at different time points and lysed using lysis buffer. Transformants were grown at 30° C. until the OD₆₀₀ reached 0.7, when recombinant protein expression was induced by the addition of IPTG to a final concentration of 0.2 mM. After 4 hours, cells were harvested by centrifugation, lysed, and centrifuged for 1 hour at 30,000 g (4° C.) to pellet the insoluble fraction. Using Qiagen's (CA, USA) Ni—NTA column and protocol, the soluble fraction containing (KH)₆-LMW—FGF2 was loaded onto a Ni—NTA column and washed with 20 volumes of wash buffer. The protein (vector) was eluted with buffer containing 1M imidazole and analyzed by western blot and sodium dodecyl sulfate polyacrylamide gel electrophoresis (SDS-PAGE). The vector was dialyzed versus Dulbecco's Phosphate Buffer Saline and stored at −80° C. after addition of 500 mM NaCl, 2 mM β-mercaptoethanol and 20% glycerin. Anti-LMW—FGF2 was used as the primary antibody to confirm the presence of LMW—FGF2 in the construct of the expressed protein (FIG. 9). The expressed protein bears a molecular weight of approximately 28,200 daltons.

The results described in this Example demonstrate the successful cloning and expression of one embodiment of the present invention of genetically engineered non-viral vectors for systemic gene delivery. This pET21b-(KH)₆-LMW—FGF2 vector encodes a gene-binding motif (K), an endosome disrupting motif (H), and a targeting ligand LMW—FGF2 LMW—FGF2 does not contain any known nuclear localization signal. It exerts its effect in the cytoplasm and is localized in the cytosol. There are a number of other pertinent publications that describe the biosynthesis of genetically engineered polymers [51, 52], their physicochemical characterization [51, 52], and in vitro and in vivo (breast and head and neck tumor models) evaluation for gene release and transfer as matrix-mediated systems [53, 54]. Other publications describe the complexation of random poly (amino acid)s with plasmid DNA, their physicochemical characterization such as gel retardation assay, size and charge measurements, and evaluation of cytotoxicity and transfection efficiency [47].

I. Amino Acid Content Analysis and MALDI-TOF

To determine the amino acid composition and exact molecular weight of the expressed protein (vector), amino acid content analysis and Matrix-Assisted Laser Desorption Ionization Mass Spectroscopy (MALDI-TOF) was performed by Commonwealth Biotechnologies Inc. (Richmond, Va., USA). For amino acid analysis, the sample was hydrolyzed in a gas phase of 6N HCl followed by drying prior to resolubilization and analysis.

J. Gel Retardation Assay

The formation of pDNA/vector complexes was examined by gel retardation. pDNA (pEGFP, Clontech, CA, USA) was complexed with (KH)₆-LMW—FGF2 at pDNA:vector molar ratios of 1:40, 1:60, 1:80, and 1:100 in 5 mM phosphate buffer saline (PBS). (KHKHKHKHKK)₅ was also synthesized (Anaspec Inc., CA, USA) and complexed with pDNA under the same conditions. Due to the limitations of current peptide synthesis technology in synthesizing repetitive peptides with low water solubility, constructs with higher KH repeats than (KH)₅ could not be synthesized; hence (KH)₅ was used as the control. In a microfuge tube, 1.2 μg of pDNA was added and diluted to 10 microliters with deionized water. Before complexation, the (KH)₆-LMW—FGF2 vector was dialyzed versus 5 mM PBS for 15 minutes and added in a separate microfuge tube to produce the desired ratio and diluted to 15 microliters with deionized water. The vector solution was added to pDNA solution and incubated at room temperature for 30 minutes. After 30 minutes, non-heat inactivated fetal bovine serum (Gemeni Bio Products, CA, USA) was added to a final concentration of 10% and the complexes were incubated for another hour at 37° C. The complexes were then electrophoresed on a 1% agarose gel and DNA was visualized by ethidium bromide staining.

K. Cell Proliferation Assay

NIH 3T3 cells were grown in F12/DMEM (1:1 ratio) with 10% fetal calf serum (FCS). At the time of assay, cells were washed with serum-free medium (F12/DMEM supplemented with insulin, transferrin, selenium, fibronectin and dexamethasone) and 5×10³ cells were seeded in a 96 well dish in 150 μl of serum-free media (SFM). A serial dilution of (KH)₅, (KH)₆-LMW—FGF2 and native LMW—FGF2 (Promega, Madison Wis., USA) were prepared across the plate ranging from 0.02 to 50 ng/ml. Cells were incubated for 44 hours at 37° C. in humidified 5% CO₂ atmosphere. After the incubation time, WST-1 (Roche Applied Science, IN, USA) reagent was added and the absorbance after 4 hours was measured at 440 nm. This assay is similar to MTS and MTT assay and is based on the reduction of WST-1 (Water Soluble Tetrazolium) by mitochondrial dehydrogenases in viable cells which produces soluble formazan dye [55]. The amount of formazan salt (dye) formed directly correlates to the number of metabolically active cells in the culture medium.

L. Cell Toxicity Assays

Cell toxicity assays were performed under two conditions: Cells incubated in SFM or in DMEM/F12 supplemented with FCS. In the first case, 5×10³ cells were seeded in a 96 well dish in 150 μl of serum-free media and incubated overnight. A serial dilution of (KH)₅ and (KH)₆-LMW—FGF2 was prepared across the plate ranging from 0.01 μg/ml to 50 μg/ml. The cells were incubated for 4 hours at 37° C. in humidified 5% CO₂ atmosphere. After the incubation time, WST-1 reagent was added and the absorbance after 4 hours was measured at 440 nm. In the second case, the same study was repeated by replacing SFM with DMEM/F12 90%, FCS 10%. The control cells were treated with Dulbecco's Phosphate Buffer Saline (DPBS) instead of (KH)₆-LMW—FGF2.

M. Photon Correlation Spectroscopy

The mean hydrodynamic sizes of plasmid DNA/copolymer complexes were determined by Photon Correlation Spectroscopy (PCS) (Malvern Zetasizer 3000, Malvern Instruments). Measurements were performed in triplicate and reported as mean±standard error, using an argon laser of 480 nm on complexes formed in water at 25° C. and an angle of 90° C. CONTIN analysis was used to fit the experimental intensity decay curve and derive the median particle diameter for the complexes.

N. Inhibition Study by LMW—FGF2

NIH 3T3 cells were seeded in 12 well tissue culture plates at 5×10⁴ cells per well in 1 ml SFM. Cells were approximately 70-80% confluent at the time of transfection. 5 μg/50 μl of pEGFP was mixed with 4.2 μg/50 μl of vector and incubated for 30 minutes at room temperature for complex formation. In one set of wells, LMW—FGF2 (1000 ng/ml) was added followed by addition of complexes. In the second set, SFM was added followed by addition of complexes (control). The cells were incubated at 37° C. in humidified 5% CO₂ atmosphere. After 4 hours, the growth media was removed and replaced with growth media supplemented with serum. Green fluorescent protein activity was visualized using a Zeiss confocal microscope.

O. Cell Culture and Transfection

NIH 3T3 cells (mouse embryo fibroblast), COS-1 cells (African green monkey kidney), and T-47D cells (human breast cancer) were propagated as suggested by the American Type Culture Collection (VA, USA). Cells were seeded in 12 well tissue culture plates (in triplicates) at 4×10⁴ cells per well in 1 ml growth media (with or without 10% FCS). Cells were approximately 60%-70% confluent at the time of transfection. 5 μg/50 μl of pEGFP (Green Fluorescent Protein) was mixed with 4.2 μg/50 μl of vector and incubated for 30 minutes at room temperature for complex formation as described above. The complexes were added to the growth media and the cells were incubated at 37° C. in humidified 5% CO₂ atmosphere. After 4 hours, the growth media was removed and replaced with growth media supplemented with serum. Green fluorescent protein activity was visualized using a Zeiss confocal microscope. From each well, three snap shots from different locations were taken and all the transfected and non-transfected cells in each snap shot were counted. Number of counted cells varied between 50-250 cells per snap shot depending on the cell density at each location. Since the samples were prepared in triplicates (three snap shot per replicate), the percent number of tranfected cells was reported as Mean±S.D. (n=9). Lipofectamine 2000 (Invitrogen, CA, USA) and (KH)₅ complexed with pEGFP were used as positive controls.

### Example 2

### Synthesis and Characterization of Genes Encoding (KHC)ₙ-LMW—FGF2.

To construct, clone and express genes encoding (KHC)ₙ-LMW—FGF2 two different sets of gene constructs are required. A set encoding lysine-histidine-cysteine (KHC) and a set encoding LMW—FGF2. In this vector, the amino acid Cysteine (C) will be engineered into the protein-based NABM polymer to facilitate release of the gene from the vector. Cysteine residues allow intracellular bioreduction and to facilitate DNA release from the protein based polymer portion of the construct. Introduction of cysteine residues in the construct has been reported to increase transfection efficiency in various cancer cell lines in comparison with cationic polymers alone [20].

A. Cloning of (KHC)₃-LMW—FGF2 in pET21b.

The pET21 b cloning/expression vector can be used in cloning and expression of the (KHC)₃-LMW—FGF2 gene. This vector adds 6× His tag to the C-terminal of inserted genes which facilitates the protein purification process by using Ni—NTA column chromatography. A cloning method will be designed to insert the genes of interest in between NdeI and HindIII restriction sites. The plasmid containing LMW—FGF2 gene can be transformed into DH5α cells and sufficient plasmid DNA will be isolated following Qiagen™ mini-prep purification protocol. The purity and concentration of purified plasmids can be measured using UV spectroscopy at 260 nm and 280 nm.

The LMW—FGF2 gene will be made by PCR amplification to produce KHC-LMW—FGF2 as shown in FIG. 11 which has proper restriction sites (NdeI, EcoRI and HindIII) to be fused with the (KHC)₃ gene. The KHC-LMW—FGF2 gene will be first digested with NdeI and HindIII and cloned into pET21b to make pET21b-KHC-LMW—FGF2.

The (KHC)₃ genes will then be cut from the pET21b-(KH)₃ vector by NdeI and EcoRI and at the same time pET21b-KHC-LMW—FGF2 will also be digested with NdeI and EcoRI in a 20 μl reaction mixture (1.5 hours, 37° C.) and loaded onto agarose gel and purified using Qiagen™ Gel Extraction kit and protocol. The (KHC)₃ gene will be cloned into pET21b-KHC-LMW—FGF2 using Quick T4 DNA Ligase (20 min, 25° C.) as described above and transformed into DH5a cells. A colony will then be grown over night to isolate enough plasmids using Qiagen™ mini-prep kit and protocol. The cloned vectors will be sequenced and insertion of both lysine-histidine-cysteine repeats and LMW—FGF2 into pET21b will be confirmed.

As an alternative to the method described above, other expression vector/hosts will be used. For example, a pET vector carrying GST tag which enhances the solubility of the protein in combination with BL21(DE3) pLysS expression host (rec A) will be used. BL21(DE3) pLysS is a expression host which has a tight control over the basal level of protein expression before induction and also is deficient in recombinase protease. Therefore, theoretically no or minimal protein will be expressed before induction or degraded after induction.

B. Expression and Protein Purification.

In one method, E. coli strain BL21 (DE3) (Novagen, Madison, Wis.) will be transformed with pET21b-(KHC)₃-LMW—FGF2, pET21b-(KHC)₃ or pET21b-LMW—FGF2 vectors. A colony will be taken and grown in LB medium. The BL21 (DE3) cells will be induced with 1 mM IPTG and cells will be harvested after 3 hours and lysed with lysis buffer. The lysate will be loaded on Ni—NTA His columns and the proteins will be purified. The over-expression of the proteins will be confirmed by SDS-PAGE and Western blot analysis. The exact molecular weight and amino acid content of expressed proteins will be determined by MALDI-TOF mass spectroscopy and amino acid content analysis, respectively.

C. Purification of Expressed Proteins.

The expressed proteins will have a 6× His tag at their C-terminal that will facilitate their purification by Ni-column chromatography. However, the His tag can be masked by the 3-D structure of the protein and its unavailability to bind to free Ni⁺ ions at the surface of Ni-column. As an alternative these proteins will either be purified with Heparin affinity column which has high affinity towards LMW—FGF2 or fused with GST tag and purified with GST-column chromatography. Any known protein isolation and purification methods may be used.

D. Attachment of (KHC)₃ by Disulfide Linkage to Constructs.

After expression of (KHC)₃ and (KHC)₃-LMW—FGF2 separately, they can be attached via disulfide bonds under oxidative polycondensation, prepared by standard Fmoc/tBoc chemistry with dimethyl sulfoxide [20]. During this process, two terminal cysteine residues at the chain ends of (KHC)₃ will form a disulfide bond. Based on this chemistry constructs (b) and (f) can be prepared. Once all the constructs are synthesized and their identity verified the next steps are complexing the vector with DNA, physicochemical characterization of the complexes and evaluation of their toxicity and transfection efficiency by established procedures [47].

E. Preparation and Evaluation of Polymer/pDNA Complexes.

Gel Retardation Assay.

The formation and net charge of the vector/pDNA (pRL CMV luc) complexes can be examined by gel retardation assay by procedure described previously [47]. Vector/pDNA complexes will be formed at different mole/mole ratios of vector/pDNA. 50 μl containing 20 μg of the plasmid DNA solution in water will be transferred into a microfuge tube. While stirring, vector solution will be added drop wise into the pDNA solution to form a complex. Complexes will be allowed to form for 30 minutes at room temperature. 20 μl of the complex solutions will be eluted on 1% agarose gel. DNA migration will be visualized by ethidium bromide staining. The vector/pDNA complexes with a net negative charge will migrate towards the positive pole. This study determines the minimum amount of vector needed to fully neutralize the negative charges at the surface of the pDNA and hence, pDNA condensation and protection from endonucleases. These studies will be done in duplicate.

F. Measurement of Zeta Potential and Particle Size.

The zeta potentials and particle size of the vector/pDNA complexes formed at different mole/mole ratios will be determined using electrophoretic light scattering technique (Malvern Zetasizer 3000, Malvern Instruments, Malvern, UK). All the glass and plastic tubing will be washed with filtered deionized water to avoid particulate contamination. For each sample, mean particle electrophoretic mobility will be measured in a thermostatically controlled microelectrophoresis cell equilibrated at 25° C. Measurements will be made in triplicates (mean±SD). This study demonstrates the net surface charge of the complexes and their particle size. Compact complexes with slightly positive, neutral, and slightly negative charges will be used in cell transfection studies.

G. Nuclease Degradation Assay.

The degradation of the pDNA in the vector/pDNA complexes in the presence of nuclease will be tested by treatment of the complexes with DNase I [47]. Vector/pDNA in Tris buffer+6 mM MgCl₂ will be incubated with RNase-free DNase I for 30 minutes at 37° C. The samples in triplicate (mean±SD) will be treated with EDTA and Heparin consecutively and analyzed by agarose gel electrophoresis. This study is conducted to evaluate the capability of the vector in preserving the pDNA from the endonuclease.

H. Stability of Vector/pDNA Complexes to Reduction.

It has been shown that a reducible polycation of sufficient molecular weight to condense DNA and the capacity to facilitate DNA release would form the basis of an effective gene delivery vector [20]. Once the complex between vector and pDNA is formed, its ability to be destabilized under reducing conditions needs to be evaluated. The effect of reduction on complexes can be examined by measuring the ability of dithiothretiol (DTT) to restore fluorescence of ethidium bromide/pDNA. In the presence of the intercalating dye ethidium bromide, fluorescence will be strongly quenched by addition of vector/pDNA complexes indicating efficient complex formation. Incubation with the reducing agent DTT will lead to an increase in fluorescence as it facilitates the disulfide bond breakage. In contrast, no change in fluorescence is expected following DTT treatment of complexes formed with non-reducible vectors such poly L-lysine. In brief, ethidium bromide will be added at a concentration of 1 μg/ml to the solution of complexes and changes in the fluorescence caused by the addition of 25 mM DTT will be measured (λ_{exc}=510 nm, λ_{cm}=590 nm).

### LITERATURE CITED

[1] Vile R G, Russell S J, Lemoine N R. Cancer gene therapy: hard lessons and new courses. Gene Ther 2000;7(1):2-8.
[2] Lynn D M, Langer R. Degradable poly(b-amino esters): Synthesis, characterization, and self-assembly with plasmid DNA. J Am Chem Soc 2000;122:10761-68.
[3] McKenzie D L, Collard W T, Rice K G. Comparative gene transfer efficiency of low molecular weight polylysine DNA-condensing peptides. J Pept Res 1999;54(4):311-8.
[4] Yi S W, Yune T Y, Kim T W, Chung H, Choi Y W, Kwon I C, et al. A cationic lipid emulsion/DNA complex as a physically stable and serum-resistant gene delivery system. Pharm Res 2000;17(3):314-20.
[5] Chen Q R, Zhang L, Stass S A, Mixson A J. Co-polymer of histidine and lysine markedly enhances transfection efficiency of liposomes. Gene Ther 2000;7(19):1698-705.
[6] Midoux P, Monsigny M. Efficient gene transfer by histidylated polylysine/pDNA complexes. Bioconjug Chem 1999;10(3):406-11.
[7] Sosnowski B A, Gonzalez A M, Chandler L A, Buechler Y J, Pierce G F, Baird A. Targeting DNA to cells with basic fibroblast growth factor (FGF2). J Biol Chem 1996;271 (52):33647-53.
[8] Cappello J. Synthetically designed protein-polymer biomaterials. In: Park K., editor. Controlled drug delivery: challenges and strategies. Washington DC, American Chemical Society, 1997: 439-53.
[9] Putnam D, Gentry C A, Pack D W, Langer R. Polymer-based gene delivery with low cytotoxicity by a unique balance of side-chain termini. Proc Natl Acad Sci USA 2001 ;98(3):1200-5.
[10] Boussif O, Lezoualc'h F, Zanta M A, Mergny M D, Scherman D, Demeneix B, et al. A versatile vector for gene and oligonucleotide transfer into cells in culture and in vivo: polyethylenimine. Proc Natl Acad Sci USA 1995;92(16):7297-301.
[11] Chen J, Stickles R J, Daichendt K A. Galactosylated histone-mediated gene transfer and expression. Hum Gene Ther 1994;5(4):429-35.
[12] Emi N, Kidoaki S, Yoshikawa K, Saito H. Gene transfer mediated by polyarginine requires a formation of big carrier-complex of DNA aggregate. Biochem Biophys Res Commun 1997;231(2):421-4.
[13] MacLaughlin F C, Mumper R J, Wang J, Tagliaferri J M, Gill I, Hinchcliffe M, et al. Chitosan and depolymerized chitosan oligomers as condensing carriers for in vivo plasmid delivery. J Control Release 1998;56(1-3):259-72.
[14] Nead M A, McCance D J. Poly-L-omithine-mediated transfection of human keratinocytes. J Invest Dermatol 1995; 105(5):668-71.
[15] Nishikawa M, Takemura S, Takakura Y, Hashida M. Targeted delivery of plasmid DNA to hepatocytes in vivo: optimization of the pharmacokinetics of plasmid DNA/galactosylated poly(L-lysine) complexes by controlling their physicochemical properties. J Pharmacol Exp Ther 1998;287(1):408-15.
[16] Perales J C, Ferkol T, Molas M, Hanson R W. An evaluation of receptor-mediated gene transfer using synthetic DNA-ligand complexes. Eur J Biochem 1994;226(2):255-66.
[17] Qin L, Pahud D R, Ding Y, Bielinska A U, Kukowska-Latallo J F, Baker J R, Jr., et al. Efficient transfer of genes into murine cardiac grafts by Starburst polyamidoamine dendrimers. Hum Gene Ther 1998;9(4):553-60.
[18] Strydom S, Van Jaarsveld P, Van Helden E, Ariatti M, Hawtrey A. Studies on the transfer of DNA into cells through use of avidin-polylysine conjugates complexed to biotinylated transferrin and DNA. J Drug Target 1993;1(2):165-74.
[19] Szoka F C, Jr. Many are probed, but few are chosen. Nat Biotechnol 1997;15(6):509.
[20] Oupicky D, Parker A L, Seymour L W. Laterally stabilized complexes of DNA with linear reducible polycations: strategy for triggered intracellular activation of DNA delivery vectors. J Am Chem Soc 2002;124:8-9.
[21] Cappello J, Crissman J, Dorman M, Mikolajczak M, Textor G, Marquet M, et al. Genetic engineering of structural protein polymers. Biotechnol Prog 1990;6(3):198-202.
[22] Fournier M J, Creel H S, Krejchi M T, Mason T L, Tirrell D A, McGrath K P, et al. Genetic synthesis of periodic protein materials. J Bioact Compat Pol 1991;6:326-38.
[23] McPherson D T, Morrow C, Minehan D S, Wu J, Hunter E, Urry D W. Production and purification of a recombinant elastomeric polypeptide, G-(VPGVG) 19-VPGV, from Escherichia coli. Biotechnol Prog 1992;8(4):347-52.
[24] Cappello J, Ferrari F. Plastics from Microbes. New York: Hanser Publishers, 1994.
[25] Quarto N, Finger F P, Rifkin D B. The NH2-terminal extension of high molecular weight bFGF is a nuclear targeting signal. J Cell Physiol 1991;147:311-18.
[26] Teule F, Aube C, Ellison A S. Production of customized novel fiber proteins in yeast (Pichiapastoris) for specialized applications. In: Proceedings of the Third International Silk Conference; 2003; Montreal, Quebec, Canada; 2003.
[27] Paul R W, Weisser K E, Loomis A, Sloane D L, LaFoe D, Atkinson E M, et al. Gene transfer using a novel fusion protein, GAL4/invasin. Hum Gene Ther 1997;8(10):1253-62.
[28] Box M, Parks D A, Knight A, Hale C, Fishman P S, Fairweather N F. A multi-domain protein system based on the HC fragment of tetanus toxin for targeting DNA to neuronal cells. J Drug Target 2003;11(6):333-43.
[29] Fominaya J, Uherek C, Wels W. A chimeric fusion protein containing transforming growth factor-alpha mediates gene transfer via binding to the EGF receptor. Gene Ther 1998;5(4):521-30.
[30] Uherek C, Fominaya J, Wels W. A modular DNA carrier protein based on the structure of diphtheria toxin mediates target cell-specific gene delivery. J Biol Chem 1998;273(15):8835-41.
[31] Cappello J, inventor Synthetic proteins for in vivo drug delivery and tissue augmentation. U.S. Pat. No. 6,380,154.
[32] Read M L, Bremner K H, Oupicky D, Green N K, Searle P F, Seymour L W. Vectors based on reducible polycations facilitate intracellular release of nucleic acids. J Gene Med 2003;5(3):232-45.
[33] Agrawal S, Zhang R. Pharmacokinetics of oligonucleotides. Ciba Found Symp 1997;209:60-75; discussion 75-8.
[34] Agrawal S, Zhao Q. Antisense therapeutics. Curr Opin Chem Biol 1998;2(4):519-28.
[35] Zhao Q, Zhou R, Temsamani J, Zhang Z, Roskey A, Agrawal S. Cellular distribution of phosphorothioate oligonucleotide following intravenous administration in mice. Antisense Nucleic Acid Drug Dev 1998;8(6):451-8.
[36] Anderson K P, Fox M C, Brown-Driver V, Martin M J, Azad R F. Inhibition of human cytomegalovirus immediate-early gene expression by an antisense oligonucleotide complementary to immediate-early RNA. Antimicrob Agents Chemother 1996;40(9):2004-11.
[37] Borchers et al., inventor Antisense modulation of hematopoietic cell protein tyrosine kinase expression describes methods for making and using antisense-oligonucleotides and their formulation. U.S. Pat. No. 6,828,151.
[38] Berger W, Setinek U, Mohr T, Kindas-Mugge I, Vetterlein M, Dekan G, et al. Evidence for a role of FGF-2 and FGF receptors in the proliferation of non-small cell lung cancer cells. Int J Cancer 1999;83(3):415-23.
[39] Chandler L A, Sosnowski B A, Greenlees L, Aukerman S L, Baird A, Pierce G F. Prevalent expression of fibroblast growth factor (FGF) receptors and FGF2 in human tumor cell lines. Int J Cancer 1999;81(3):451-8.
[40] McLeskey S W, Zhang L, Kharbanda S, Kurebayashi J, Lippman M E, Dickson R B, et al. Fibroblast growth factor overexpressing breast carcinoma cells as models of angiogenesis and metastasis. Breast Cancer Res Treat 1996;39(1):103-17.
[41] Tannheimer S L, Rehemtulla A, Ethier S P. Characterization of fibroblast growth factor receptor 2 overexpression in the human breast cancer cell line SUM-52PE. Breast Cancer Res 2000;2(4):311-20.
[42] Urry D W, Harris C M, Luan C X, Luan C-H, Channe-Gowda D, Parker T M, et al. Transductional protein-based polymers as new controlled-release vehicles. In: Park K., editor. Controlled drug delivery: challenges and strategies. Washington, DC, American Chemical Society, 1997: 405-38.
[43] Wyman T B, Nicol F, Zelphati O, Scaria P V, Plank C, Szoka F C, Jr. Design, synthesis, and characterization of a cationic peptide that binds to nucleic acids and permeabilizes bilayers. Biochemistry 1997;36(10):3008-17.
[44] Chan C K, Jans D A. Using nuclear targeting signals to enhance non-viral gene transfer. Immunol Cell Biol 2002;80(2):119-30.
[45] Escriou V, Carriere M, Scherman D, Wils P. NLS bioconjugates for targeting therapeutic genes to the nucleus. Adv Drug Deliv Rev 2003;55(2):295-306.
[46] Munkonge F M, Dean D A, Hillery E, Griesenbach U, Alton E W. Emerging significance of plasmid DNA nuclear import in gene therapy. Adv Drug Deliv Rev 2003;55(6):749-60.
[47] Haider M, Ghandehari H. Influence of poly (amino acid) composition on complexation with plasmid DNA and transfection efficiency. J Bioact Compat Pol 2003;11 :93-111.
[48] McMillan R A, Lee T A T, Conticello V P. Rapid assembly of synthetic genes encoding protein polymers. Macromolecules 1999;32:3643-48.
[49] Brown M D, Gray A I, Tetley L, Santovena A, Rene J, Schatzlein A G, et al. In vitro and in vivo gene transfer with poly(amino acid) vesicles. J Control Release 2003;93(2):193-211.
[50] Peyrat J P, Bonneterre J, Hondermarck H, Hecquet B, Adenis A, Louchez M M, et al. Basic fibroblast growth factor (bFGF): mitogenic activity and binding sites in human breast cancer. J Steroid Biochem Mol Biol 1992;43(1-3):87-94.
[51] Nagarsekar A, Crissman J, Crissman M, Ferrari F, Cappello J, Ghandehari H. Genetic synthesis and characterization of pH- and temperature-sensitive silk-elastinlike protein block copolymers. J Biomed Mater Res 2002;62(2):195-203.
[52] Nagarsekar A, Crissman J, Crissman M, Ferrari F, Cappello J, Ghandehari H. Genetic engineering of stimuli-sensitive silkelastin-like protein block copolymers. Biomacromolecules 2003;4(3):602-7.
[53] Megeed Z, Cappello J, Ghandehari H. Controlled release of plasmid DNA from a genetically engineered silk-elastinlike hydrogel. Pharm Res 2002;19(7):954-9.
[54] Megeed Z, Haider M, Li D, O'Malley B W, Jr., Cappello J, Ghandehari H. In vitro and in vivo evaluation of recombinant silk-elastinlike hydrogels for cancer gene therapy. J Control Release 2004;94(2-3):433-45.
[55] Liu S Q, Saijo K, Todoroki T, Ohno T. Induction of human autologous cytotoxic T lymphocytes on formalin-fixed and paraffin-embedded tumour sections. Nat Med 1995;1(3):267-71.

What is claimed is:

1. A genetically engineered non-viral vector for delivering a nucleic acid molecule to a target cell, comprising a nucleic acid-binding protein-based polymer comprising at least one tandem repeat of a cationic amino acid-containing monomer which monomer is capable of binding to the nucleic acid molecule.

2. The genetically engineered non-viral vector of claim 1, wherein the cationic amino acid-containing monomer comprises one or more amino acids selected from the group consisting of lysine and arginine.

3. The genetically engineered non-viral vector of claim 2, wherein the cationic amino acid-containing monomer comprises from about 10% to about 70% of one or more amino acids selected from the group consisting of lysine and arginine.

4. The genetically engineered non-viral vector of claim 2, wherein the cationic amino acid-containing monomer comprises from about 30% to about 60% of one or more amino acids selected from the group consisting of lysine and arginine.

5. The genetically engineered non-viral vector of claim 2, wherein the cationic amino acid-containing monomer further comprises histidine.

6. The genetically engineered non-viral vector of claim 2, wherein the cationic amino acid-containing monomer comprises from about 10% to about 70% histidine.

7. The genetically engineered non-viral vector of claim 2, wherein the cationic amino acid-containing monomer comprises from about 20% to about 40% histidine.

8. The genetically engineered non-viral vector of claim 1, wherein the cationic amino acid-containing monomer further comprises amino acids having imidazole side chains.

9. The genetically engineered non-viral vector of claim 2, wherein the cationic amino acid-containing monomer further comprises one or more cysteine residues.

10. The genetically engineered non-viral vector of claim 1, further comprising a protein or peptide targeting ligand that is recognized by a target cell.

11. The genetically engineered non-viral vector of claim 10, wherein the targeting ligand binds to a receptor expressed on the surface of the target cell.

12. The genetically engineered non-viral vector of claim 10, wherein the targeting ligand is an antibody that recognizes an antigen on the surface of the target cell.

13. The genetically engineered non-viral vector of claim 11, wherein the targeting ligand is fibroblast growth factor 2 (FGF2) or a fragment thereof.

14. The genetically engineered non-viral vector of claim 10, wherein the target cell is a cancer cell.

15. The genetically engineered non-viral vector of claim 14, wherein the cancer cell expresses fibroblast growth factor 2 (FGF2) receptor.

16. The genetically engineered non-viral vector of claim 14, wherein the cancer cell is selected from the group comprising ovarian cancer, breast cancer, colon cancer, and lung cancer.

17. The genetically engineered non-viral vector of claim 1, further comprising a nuclear localization sequence.

18. The genetically engineered non-viral vector of claim 1, further comprising an endosome disrupting moiety.

19. The genetically engineered non-viral vector of claim 18, wherein the endosome disrupting moiety is histidine.

20. The genetically engineered non-viral vector of claim 1, wherein the vector is bound to the nucleic acid molecule forming a vector/nucleic acid complex.

21. The genetically engineered non-viral vector of claim 1, wherein the vector is transcribed from a single gene.

22. The genetically engineered non-viral vector of claim 1, wherein the cationic amino acid-containing monomer is selected from the group comprising SEQ ID NO. 1, SEQ ID NO. 8 and SEQ ID NO. 9.

23. The genetically engineered non-viral vector of claim 1, comprising more than one different cationic amino acid-containing monomer.

24. The genetically engineered non-viral vector of claim 1, selected from the group comprising SEQ ID NO. 3, SEQ ID NO. 4, SEQ ID NO. 5, SEQ ID NO. 6, and SEQ ID NO. 12.

25. A method for delivering a nucleic acid molecule to a target cell, comprising
a) obtaining a genetically engineered non-viral vector comprising a nucleic acid-binding protein-based polymer that contains at least one tandem repeat of a cationic amino acid-containing monomer capable of binding to the nucleic acid molecule;
b) contacting the vector of step a with the nucleic acid molecule under conditions that permit the vector to bind to the nucleic acid molecule to form a complex;
c) contacting the vector/nucleic acid molecule complex of step b with the target cell under conditions that permit the vector/nucleic acid molecule complex to be internalized by the target cell.

26. The method of claim 25, wherein the cationic amino acid-containing monomer comprises one or more amino acids selected from the group consisting of lysine and arginine.

27. The method of claim 25, wherein the cationic amino acid-containing monomer is a homopolymer of an amino acid selected from the group consisting of lysine and arginine.

28. The method of claim 25, wherein the cationic amino acid-containing monomer comprises from about 10% to about 70% of one or more amino acids selected from the group consisting of lysine and arginine.

29. The method of claim 25, wherein the cationic amino acid-containing monomer comprises from about 30% to about 60% of one or more amino acids selected from the group consisting of lysine and arginine.

30. The method of claim 25, wherein the vector further comprises a targeting ligand that is recognized by the target cell.

31. The method of claim 25, wherein the vector further comprises a nuclear localization sequence that permits the vector/nucleic acid molecule complex to enter the nucleus of the target cell.

32. The method of claim 31, wherein the vector further comprises an endosome disrupting moiety.

33. The method of claim 25, wherein the target cell is an animal cell.

34. The method of claim 25, wherein target cell is a cancer cell.

35. The method of claim 34, wherein the cancer cell is selected from the group comprising ovarian cancer, breast cancer, colon cancer, and lung cancer.

36. The method of claim 34, wherein the cancer cell expresses fibroblast growth factor receptor 2 (FGF2) on its surface.

37. The method of claim 25, wherein the target cell is a bacterial cell.

38. The method of claim 25, wherein the target cell is a plant cell or a bacterial cell.

39. A genetically engineered non-viral vector for delivering a nucleic acid molecule to a target cell, comprising a member of the group consisting of homolysine, homoarginine, and copolymers of lysine-histidine, arginine-lysine, arginine-histidine or lysine-arginine-histidine.

40. A complex comprising a genetically engineered non-viral vector bound to a nucleic acid molecule, wherein the vector is intended for delivering the nucleic acid molecule to a target cell, and the vector comprises a nucleic acid-binding protein-based polymer comprising at least one tandem repeat of a cationic amino acid-containing monomer which monomer is capable of binding to the nucleic acid molecule.

41. The vector of claim 1, wherein the nucleic acid is DNA or RNA.

42. The complex of claim 40, wherein the nucleic acid is DNA or RNA

43. The vector of claim 1, wherein the RNA is antisense RNA.

44. The complex of claim 40, wherein the RNA is antisense RNA.

45. A pharmaceutical composition for gene therapy comprising, a complex comprising a genetically engineered non-viral vector bound to a therapeutic gene, wherein the vector comprises a nucleic acid-binding protein-based polymer comprising at least one tandem repeat of a cationic amino acid-containing monomer which monomer is capable of binding to the nucleic acid molecule.

46. The composition of claim 45, wherein the vector further comprises a targeting ligand.

47. The composition of claim 46, wherein the targeting ligand is recognized by a cancer cell.

48. The composition of claim 47, wherein the therapeutic gene is delivered to the cancer cell.

49. The composition of claim 45, wherein the targeting ligand is FGF2.

50. The genetically engineered non-viral vector of claim 1, suitable for systemic administration to an animal.
